(19) United States
(12) Patent Application Publication
(10) Pub. No.: US 2008/0255624 A1
(43) Pub. Date: Oct. 16, 2008

(54) Methods and devices for multipoint access of a body part

(76) Inventors: Gregory Arcenio, Redwood City, CA (US); Andrew C. Kohm, Burlingame, CA (US)

Correspondence Address: HAYNES AND BOONE, LLP, 901 Main Street, Suite 3100, Dallas, TX, 75202, US

(21) Appl. No.: 11/731,825

(22) Filed: Mar. 30, 2007

(51) Int. Cl. A61F 5/00; A61B 17/70; A61M 29/00; A61B 17/00

(52) U.S. Cl. 606/86 R; 606/87; 606/192; 606/82

(57) ABSTRACT

Disclosed are methods and devices for accessing body parts using a plurality of access points to facilitate the repair of such body parts. The multipoint access devices and methods of the present invention may facilitate manipulating tools and materials that can be used in the repair of bone and other body parts. In certain embodiments, the multipoint access and repair devices of the present invention may be used for sectioning and repair of a vertebral body or an intervertebral disc. Additionally, methods and devices for emplacing an expandable barrier structure are disclosed.

### CROSS REFERENCE TO RELATED APPLICATIONS

This application is related to application, KY-256, entitled Devices For Multipoint Emplacement In A Body Part And Methods Of Use Of Such Devices, filed on Mar. 30, 2007.

### FIELD OF INVENTION

The present invention relates to methods and devices for multipoint access of a body part as well as devices for multipoint emplacement in a body part and methods of use of such devices.

### BACKGROUND

There are often instances where a body part must be accessed from a plurality of separate entry points. For example, when accessing a spine, it may be beneficial to access the vertebral body using two extrapedicular cannulas as a means to avoid the spinal cord, vasculature or other organs.

An example of this type of procedure is the repair of spinal fractures. In many spinal fractures, the vertebral body heals in a fracture-induced position, such that the upper and lower endplates of the vertebral body are no longer parallel, resulting in curvature of the spine. To correct the shape of the spine, it may be necessary to refracture the vertebral body so as to insert a bone repair material. Such refracturing may be done by sectioning the vertebral body axially to at least partially split the vertebral body into a superior and an inferior portion. However, in older vertebral body wedge fractures, it may be difficult to section the vertebral body due to the presence of the hardened sclerotic bone which can be very difficult to curette and/or fracture.

Also, in cases where the body part has a surface that is no longer intact, there may be a need to emplace a repair material and/or a device in a body part to functionally replace the damaged structure. However, for a body part being repaired by the introduction of a repair material, there may be a need to emplace a device that can serve as a barrier to prevent the repair material from leaking out of the damaged area. For example, in the case of repairing a spinal fracture, once the vertebral body is sectioned, there may be a gap created in a portion of the wall of the vertebral body. Complete repair of the fracture may require introduction of a bone repair material into the vertebral body to achieve endplate parallelism. If there is a gap in the vertebral wall, the repair material can leak out of the gap in certain situations.

### SUMMARY

Embodiments of the present invention comprise methods and devices for multipoint access of a body part. In certain embodiments, the body part may comprise a bone or bone tissue. For example, the body part may comprise a spinal tissue such as a vertebral body and an intervertebral disc.

The present invention may be embodied in a variety of ways. Certain embodiments may comprise a method to access an internal body part using a device comprising a plurality of access paths or access members. In one embodiment, the access to the body part is bilateral. For example, the method may comprise the steps of: (a) accessing a body part via a first entry point using a first access member; (b) accessing the body part via a second entry point using a second access member; (c) positioning the first and second access members such that the distal ends of each access member are in position to communicate with each other. The method may further comprise establishing a communication between the two access members, such that the two access members function in a coordinated manner. Yet other embodiments of the present invention may comprise devices for accessing a body part by a plurality of access points.

Other embodiments include placing a device or tool into a spinal tissue inside a patient's body through a first access path or access member, and controlling the device within the spinal tissue through cooperation between the first access path or access member and a second access path or access member. The access paths may be percutaneous paths established via access members such as cannulas. In one example, the distal end of a first elongated member may be connected to a device. The device may be inserted into a spinal tissue (e.g., vertebral body, intervertebral disc, etc.) within a patient's body through a first percutaneous path, which provides access to the spinal tissue. A distal portion of a second elongated member may be inserted into the spinal tissue through a second percutaneous path, which provides access to the spinal tissue. The distal portion of the second elongated member may be connected to the device inside the spinal tissue, thereby establishing communication between the two percutaneous paths. By operating a proximal portion of the first elongated member and a proximal portion of the second elongated member, the user may cooperatively control the device within the spinal tissue. In one embodiment, as for example where the access path comprises and access member, the first and second elongated members may comprise inner members that fit within the access members. In one variation, the device may be used to manipulate the spinal tissue (e.g., cut or remove tissue from the interior of the spinal tissue, etc.). In another variation, the position and/or configuration of the device may be altered while located within the spinal tissue. Optionally, the device may be implanted inside the tissue. For example, the user may disconnect both the first elongated member and the second elongated member from the device and remove both elongated members from the patient's body while leaving the device inside the spinal tissue.

Embodiments of the present invention may also comprise methods and devices for emplacement of an expandable barrier device in a body part. For example, the method may comprise emplacing the expandable device into the body part via at least a first access member. The method may further comprise positioning the expandable device within the body part via the first access member and a second access member that is in communication with the first access member. In one embodiment, the method may also comprise expanding the expandable device. In an embodiment, the expandable device may be expanded in situ, to seal an aperture in the body part.

Other embodiments and further details on various aspects of the present invention are set forth in the following description, figures, and claims. It is to be understood that the invention is not limited in its application to the details set forth in the following description, figures, and claims, but is capable of other embodiments and of being practiced or carried out in various ways.

### BRIEF DESCRIPTION OF THE FIGURES

FIG. 1 shows a top view of a bialateral access of a vertebral body by first and second access paths created using first and second access members (e.g., cannulas) in accordance with one embodiment of the present invention.

FIG. 2 shows side view of a bilateral access of a vertebral body in accordance with one embodiment of the present invention.

FIG. 3 shows a top view of a curette being used to cut away a portion of the center of a vertebral body so as to establish access between two cannulas in accordance with one embodiment of the present invention.

FIG. 4 shows a top view of a first cannula into which is inserted a first inner member (e.g., wire saw) having a ferrule tip at the distal end, and a second cannula into which is inserted a second inner member (e.g., rod) having a magnet (e.g., a rare earth magnet) at the distal end in accordance with one embodiment of the present invention.

FIG. 5 shows a top view of a ferrule tip on the distal end of a wire saw engaging a magnet on the distal end of a rod such that the wire saw is pulled from the first cannula into the second cannula in accordance with one embodiment of the present invention

FIG. 6 shows a top view of a wire saw pulled completely through a first catheter and a second cannula in accordance with one embodiment of the present invention.

FIG. 7 shows a top view of a first inner member (e.g., wire saw) that may be pulled back and forth to cut out a portion of a vertebral body in accordance with one embodiment of the present invention.

FIG. 8 shows a top view of a vertebral body where additional material removed by a first inner member (e.g., wire saw) that has been pulled back and forth to cut out a portion of a vertebral body in accordance with one embodiment of the present invention.

FIG. 9 shows a side view of a first inner member comprising a wire saw that has been pulled back and forth to cut out a portion of a vertebral body in accordance with one embodiment of the present invention.

FIG. 10 shows a bialateral access of an intervertebral disc by first and second access paths created using first and second access members (e.g., cannulas) in accordance with one embodiment of the present invention where panel A shows an isometric view, panel B shows a cross-sectional view of two cannulas inserted into the disc, and panel C shows a cross-sectional view of a tool inserted into one cannula and a stylet in the other cannula; and panel D shows the tool positioned in the disc.

FIG. 11 shows balloons inserted into a vertebral body via two cannulas and being used to separate a partially sectioned vertebral body into a superior (upper) and inferior (lower) portion, where panel A is a top view of the balloons as partially inflated, panel B is a top view of the balloons more fully inflated, and panel C is a side view of the balloons more fully inflated, in accordance with alternate embodiments of the present invention.

FIG. 12 shows a diagrammatic representation of a method for establishing a bilateral access in a vertebral body in accordance with one embodiment of the present invention.

FIG. 13 shows a side view of a vertebral body comprising a gap needing repair.

FIG. 14 shows a top view of a bialateral access of a vertebral body having a gap on the anterior side of the vertebral body in accordance with one embodiment of the present invention.

FIG. 15 shows a top view of an inflatable member inserted into a vertebral body using a bilateral access in accordance with alternate embodiments of the present invention, where panel A shows the expandable member having a ferrule tip on its distal end inserted into the first access member cannula and a rod (e.g., stylet) having a magnet inserted into the second access member cannula where the ferrule tip is binding the magnet; panel B shows the rod being used to pull the expandable member through the second cannula; and panel C shows the expandable member positioned to extend from the distal ends of the first and second cannulas.

FIG. 16 shows an inflatable member being expanded to provide a support for the anterior portion of a vertebral body using a bilateral access in accordance with one embodiment of the present invention wherein panel A shows a top view and panel B shows a side view.

FIG. 17 shows examples of expandable barriers being expanded in accordance with alternate embodiments of the present invention, where panels A, B, and C show the use of a single wire to expand an expandable barrier, and panel D shows a cross-sectional side view of the expanded barrier in a body part; panels E and F show the use of a liquid or a gas to expand an expandable barrier, and panel G shows a cross-sectional side view of the inflated expanded barrier in a body part; and panels H and I show the use of a wire framework to expand a membrane expandable barrier, and panel J shows a cross-sectional side view of the expanded membrane barrier in a body part; panel K shows a two piece barrier connected at a place where the two barriers overlap, and panel L shows a two piece barrier connected where the two pieces meet.

FIG. 18 shows a top view of an expandable barrier expanded to provide a support for the anterior portion of a vertebral body and a material being inserted into a void in the vertebral body, where panel A shows the material being introduced, and panel B shows the void filled with material in accordance with alternate embodiments of the present invention.

FIG. 19 shows a method for emplacing an expandable barrier in accordance with one embodiment of the present invention.

FIG. 20 shows a kit comprising devices for multipoint access of a body part in accordance with one embodiment of the present invention.

FIG. 21 shows a device for sectioning a vertebral body in accordance with an embodiment of the present invention.

FIG. 22 shows fluoroscopic imaging of a device of the present invention being used to cut away the interior of a vertebral body where panel A shows a top view and panel B shows a side view in accordance with alternate embodiments of the present invention.

### DETAILED DESCRIPTION

Unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Also, where ranges are provided, it is understood that other embodiments within the specified ranges are to be included.

As used herein, a subject is an animal. For example, the subject may comprise a mammal. In one embodiment, the subject may be a human. The user of the products, methods, and systems of the present invention may be a physician, veterinarian, a health care professional, or another person or device.

As used herein, an internal body part may comprise a bone, bony tissue (e.g., spinal tissue), or bones or part of a bone. The body part may comprise a portion of a spine, such as a vertebral body or an intervertebral spinal disc. For example, due to various traumatic or pathologic conditions, such as cancer, a vertebral body or an intervertebral disc can experience fracture, degradation of the tissue, or expansion, and the like, that can lead to compression of the nerves, a reduction in mobility, and discomfort. The present invention is not, however, limited in application to bones, discs, or vertebrae, and may be used to repair other parts of a living or non-living organism. For example, in embodiments, the devices, methods, and systems or kits of the present invention can be deployed in other bones or other tissue types, such as an arm bone, a leg bone, an organ (e.g., stomach or other organs needing emplacement of therapeutic linings that may be expanded in situ), a portion of the vasculature, cartilage or tendons requiring surgical access and repair (e.g., a joint), and other body parts.

As used herein, an access path is an incision made in a subject to access an internal body part. In an embodiment, a percutaneous access is used. As used herein, a percutaneous access is a procedure whereby access to an inner organ or tissue is done via needle puncture of the skin, rather than using an “open” approach where the inner organs or tissue are exposed (e.g., such as surgery or cutting the skin with a scalpel). In an embodiment, the access path is made using an access member. For example, a percutaneous surgical access denotes passage through substantially unbroken skin, as for example, by needle puncture, a cannula or a catheter.

Also, as used herein, an access member comprises a device for accessing a predetermined location in a subject. The inner volume of the access member may provide a path to access a region or a body part that is located within the subject's body. The access member may be any type of device that can extend from the location of interest (e.g., a bone or an organ) to be accessible to a user of the access member. For example, the access member may be designed to extend from an internal body part (e.g., a spine or other type of bone) in a subject to outside of the subject's body. As described in more detail herein, the access member may be an elongated hollow member such as a hollow cylinder, a tube, a cannula or a delivery catheter.

Also, as used herein, a material for emplacement within, or delivery to, a body part in a subject may comprise any material that is biologically compatible with the body part of interest. For example, in alternate embodiments, the material may comprise a bone filler material or an adhesive. As used herein, a bone filler material comprises any material that may be used for the treatment of bone. A variety of materials have been described for use as bone filler materials (see e.g., U.S. Pat. Nos. 4,904,257, 6,203,574, 6,579,532, 6,740,093, and Patent Application No. 2005/0136038 for descriptions of bone filler materials). In one embodiment, the bone filler or treatment material may comprise PMMA. Alternatively, the bone filler material may comprise a cement, a gel, a fluid, or an adhesive, such as materials that are commercially available for repair of the spine and other bones or boney tissues.

The words “anterior” and “posterior” refer to the front and back of the subject, respectively. In addition, the words “proximal” and “distal” refer to direction closer to and away from, respectively, an operator (e.g., surgeon, physician, nurse, technician, etc.) who would insert the device of the present invention into the subject, with the tip-end (i.e., distal end) of the device inserted inside a subject's body. Thus, for example, the end of the access member inserted inside the subject's body would be the distal end of the access member, while the end of the access member outside the subject's body would be the proximal end of the access member.

As used herein, the term “communication” refers to the ability of two separate entities to be able to function together. For example, in one embodiment, a third member may be used to functionally connect the ends of two separate access members. It is understood that to establish communication between the two access members, the two access member may, but do not necessarily, have to come into contact with each other. In one embodiment, the two access members do not come into contact within each other inside the body part. For example, in one embodiment, the distal portion of an elongated member is inserted into the body part through the first access member. The distal portion of the elongated member is then drawn into the lumen of the second access member through the distal opening of the second access member, and thereby communication is established between the two access members. The elongated member may be cooperatively controlled through the two access members, and utilized to alter the structure of the body part.

As used herein, a bilateral access is such that the body part is accessed from both sides of a midline along at least one plane (e.g., the x-y plane or the x-z plane).

Also as used herein, an elongated member is a member that is substantially longer in length than in diameter or circumference. Examples of elongated members are cannulas, stylets and wires.

### Multipoint Access of a Body Part

Embodiments of the present invention may be used to provide a multipoint access to one or more of a variety of body parts. In one embodiment, the use of a multipoint access enhances the ability of an operator (e.g., a surgeon) to manipulate a tool or to emplace a repair material in the body part. In some embodiments, the body part of interest may comprise a bone or portion of a bone. For example, in one embodiment, the body part may comprise a vertebral body. In another embodiment, the body part comprises an intervertebral disc.

For example, due to various traumatic or pathologic conditions, such as osteoporosis, a vertebral body can experience a vertebral compression fracture (VCF). In such conditions, at least a part of the vertebral bone can be compacted, causing a decrease in height of the vertebra. In many cases, vertebral height is lost in the anterior region of the vertebral body. Thus, the products, methods, kits and systems of the present invention may by used to repair a vertebral body lost due to a fracture, or when other degeneration occurs. Or, the products, methods, kits and systems of the present invention may by used to repair an intervertebral disc as for example, when degeneration of the disc occurs. The present invention is not limited in application to spinal discs and vertebrae, and may be used to repair other parts of a living or non-living organism. For example, in embodiments, the products, methods, kits and/or systems of the present invention can be deployed in other bone types and within or adjacent to other tissue types, such as an arm bone, a leg bone, a knee joint, and the like.

For example, in certain embodiments, the present invention may comprise a method to access the interior of an internal body part. In one embodiment, the method may comprise establishing an access path, or a plurality of access paths to the body part. In one embodiment, the method may comprise using a device comprising a plurality of access members. For example, the access paths may be percutaneous paths established via an access member such as a cannula. In one embodiment, the access to the body part may be bilateral.

For example, in certain embodiments, the method may comprise the steps of: (a) accessing a body part via a first entry point using a first access member; (b) accessing the body part via a second entry point using a second access member; (c) positioning the first and second access member such that the distal ends of each access member are in position to communicate with each other; and (d) establishing a communication between the two access members, such that the two access members function in a coordinated manner. In one embodiment, the first and second access members are positioned within the interior of the body part so as to provide access to a portion of the body part needing therapy or repair.

Other embodiments include methods for placing a device or tool into a spinal tissue inside a patient's body through a first access path, and controlling the device within the spinal tissue through cooperation between the first access path and a second access path. The access paths may be percutaneous paths established via access members such as cannulas. In one example, the device may comprise a first elongated member at the distal end of the device. The device may be inserted into a spinal tissue (e.g., a vertebral body, an intervertebral disc, etc.) within a patient's body through a first percutaneous path, which provides access to the spinal tissue. A distal portion of a second elongated member may be inserted into the spinal tissue through a second percutaneous path, which provides access to the spinal tissue. The distal portion of the second elongated member may be connected to the distal end of the device inside the spinal tissue, thereby establishing communication between the two percutaneous paths. In an embodiment, the first and second elongated members may comprise first and second inner members. The first and second inner members may be sized to fit within the first and second access members.

As described herein, by operating a proximal portion of the first elongated member and a proximal portion of the second elongated member, the user may cooperatively control the device within the spinal tissue. In one embodiment, the device may be used to manipulate the spinal tissue (e.g., cut or remove tissue from the interior of the spinal tissue, etc.). In another embodiment, the position and/or configuration of the device may be altered while located within the spinal tissue. For example, the device may be positioned near an aperture in the body part, and expanded in situ to seal the aperture. Optionally, the device may be implanted inside the tissue. For example, the user may disconnect both the first elongated member and the second elongated member from the device and remove both elongated member from the patient's body while leaving the device inside the spinal tissue.

Embodiments of the present invention also comprise devices for multipoint access of a body part. In certain embodiments, the device may comprise a first access member and a second access member. The device may also comprise a first inner member to be inserted into the first access member, and a second inner member to be inserted into the second access member. In one embodiment, the first and/or second inner members may comprise an elongated member(s). In one embodiment, the distal end of the first inner member comprises a first binding component that can engage a second binding component on the distal end of the second inner member. In some embodiments, the first or second inner members may comprise a device to be implanted in the body part. For example, the device may comprise a first elongated member that can be connected to the distal portion of the second inner member thereby establishing communication between the two access members in situ. In on embodiment, the second inner member may comprise a stylet.

As described herein, the access path may comprise a percutaneous access to the spine. For example, the first and second access members may be configured for accessing the spine. In certain embodiments, the first and second access members are configured for accessing the vertebral body. Or, the first and second access members are configured for accessing the intervertebral disc. The first and second members may be configured to access the spine by percutaneous access. Where the body part is a vertebral body, the two entry points may be either transpedicular or extrapedicular. Thus, in one embodiment, the first access member may be configured for accessing interior of a vertebral body through a first pedicle of the vertebral body, and the second access member may be configured for accessing interior of the vertebral body through a second pedicle of the vertebral body. Once the access members are inserted into the body part, the distal ends of the first and second access members may be positioned adjacent to a portion of the body part requiring sectioning.

There may be a variety of approaches whereby a communication is established between the two access members or access paths. In certain embodiments, the step of establishing a communication between the two access members or access paths may comprise: (i) passing a first inner member through the first access member or access path; (ii) passing a second inner member through the second access member or access path; and (iii) engaging the distal end of the first inner member with the distal end of the second inner member such that the distal end of the first inner member binds to the distal end of the second inner member. In this way, the first and second inner members may be connected, so as to be in communication with each other and to establish a functional communication between the two access members or access paths. In one embodiment, the first inner member and the second inner member are engaged in such a manner so as to function as a single member. As described herein, the access path may comprise a percutaneous access path. Additionally or alternatively, the access member may comprise a cannula, as for example, where the body part is a vertebral body or an intervertebral disc.

There may be a variety of methods whereby the first and second inner members may be connected or engaged. In one embodiment, the distal end of the first inner member comprises a fixture that is able to bind to a fixture on the distal end of the second inner member. In one embodiment, the end of one inner member may comprise a magnet (e.g., a rare earth magnet), and the end of the other inner member may comprise a material that binds to a magnet. For example, in one embodiment, the distal end of the second inner member may comprise a magnet, and the distal end of the first inner member may comprise a material that binds to the magnet. In one embodiment, the distal end of the first inner member comprises a ferrous material. Or, in one embodiment, the distal end of the first inner member may comprise a magnet and the distal surface of the second inner member may comprise a material that binds the magnet. Or, the distal end of both inner members may comprise a magnet or rare earth magnet.

In other embodiments, a fastening device may be used to engage the two inner members and establish a communication between the first and second access members. For example, the distal end of one inner member may comprise a hook and the distal end of the other inner member may comprise a loop. Or, the distal end of one inner member may comprise a protrusion (e.g., a male fitting) that fits within an aperture or well (e.g., a female fitting) on the end of the other inner member. Or, in one embodiment, one of the inner members may comprise a micro-rongeur that is able to couple the distal end of the other inner member.

Another embodiment may have the second inner member take the form of a collapsible claw or basket. This claw can be made from aluminum, stainless steel, spring steel, nickel titanium alloys, or other alloys. Or, in some embodiments, the claw may be made of plastic. For example, a resilient plastic such as vinyl, nylon, polypropylene, a polyethylene, ionomer, polyurethane, and polyethylene tetraphthalate (PET) may be used. The first inner member may be comprised of a flexible, string-like member and have a ferrule, possibly spherical in shape, bonded to its distal end.

For example, where a claw and ferrule are used, both the first and second inner members may be advanced down their respective access members such that ferrule on the distal end of the first inner member would lie within or pass through the jaws of the claw of the second inner member. Once the inner members are in this position, the basket may be collapsed around the distal end of the first inner member. For example, a sheath may be translated over the second inner member thereby collapsing the claw around the ferrule. Or, the basket may comprise an actuating mechanism, such as a wire, that when pulled by an operator, collapses the basket. With the ferrule and accompanying first inner member captured within the claw of the second inner member, the second inner member may be pulled back through the second access member by pulling the first inner member up and through the second access member.

It will be understood that the designation of first inner member and second inner member are non-limiting and that the two members may be interchanged such that the first inner member may comprise a magnet, female aperture, or claw and the second inner member may comprise a ferrule or male device. One of ordinary skill in the art having the benefit of this disclosure would appreciate that the methods to establish communication disclosed above may also be configured to in additional manners using physical connectors or chemical bonding agents known in the art.

Once a communication is established between the two access members or the two access paths, the operator (e.g., physician or surgeon) may be able to use the two access members or access paths to manipulate a tool for the repair or alteration of the body part. Or, the operator may be able to use the two access members or access paths to insert an inflatable member(s) (e.g., kyphoplasty balloon(s)) to further shape the interior of the body part. Or, the operator may be able to use the two access members or access paths to emplace a repair material in the body part. Or, the operator may be able to use the two access members or access paths to repair a hole in the body part by emplacement of an expandable member as described herein. Again, one of ordinary skill in the art having the benefit of this disclosure would appreciate that the methods to establish communication disclosed above may be used to allow for repair or alteration of the body part using tools and devices that are known in the art.

The inner members may comprise a variety of embodiments, depending upon the particular application for which the methods, systems or devices of the present invention are being used. In certain embodiments, the first inner member is used to establish a connection between the two access members. In certain embodiments, the inner member comprises an elongated member. For example, in a embodiment, the first inner member may comprise a wire that functionally connects the distal ends of the two access members. In this way, the operator may be able to manipulate the proximal ends of the access member so that the distal ends of the access members function in a coordinated fashion. For example, in one embodiment, a first member (e.g. wire) may be threaded through a first access path or a first access member (e.g., cannula) and back up through a second access path or a second access member cannula such that the first member runs from the proximal to the distal end of the first cannula and back up from the distal end to the proximal end of the second cannula. At this point, the operator may manipulate the distal ends of the two cannulas as a single tool. For example, in one embodiment, the first inner member may comprise a leader wire that is positioned distal to a tool. The leader wire may thus be threaded down the first access member and into the second access member as a way of allowing an operator to control the tool using both access members.

The communicating access members (or access paths) may be used for a variety of tasks. In one embodiment, the two access paths or two access members may be used for the introduction of a repair material. Or, the two access paths or access members may be used for the introduction of a laser beam. For example, a laser beam may be directed into the body part for surgical repair of the body part. Or, the two access paths or access members (e.g. cannulas) may be used for the introduction of a repair tool.

In some embodiments, the first inner member may comprise a tool to modify or repair the body part. For example, the first inner member may comprise a cutting tool. In one embodiment, the first member may comprise a wire saw known as a giggly saw. In one embodiment, the wire saw may be used to cut through or section a portion of the body part. In another embodiment, the first inner member may comprise an expandable barrier as described in further detail below.

In one embodiment, an inner member may comprise a material that is substantially rigid. For example, in certain embodiments, at least one inner member may comprise a rigid rod or a stylet. Or, the inner member may be bendable. For example, in one embodiment, at least one inner member may comprise a wire saw. Or, the inner member may also comprise portions that are substantially rigid combined with portions that are bendable.

In certain embodiments, the inner member(s) may comprise a material that comprises structural or shape “memory.” In one embodiment, a shape memory material, such as nitinol, may be used. As is known in the art, a shape memory material may be urged from a first shape to a second shape by the application of external energy, but when the external energy is removed, the material will resume its original shape without loss of strength or internal structure. For example, one can bend a straight wire that is made of a shape memory alloy, and upon removing the force required to bend the wire, the wire will resume its straight conformation. As is known in the art, such shape memory materials are commercially available in various compositions, conformations, surface finishes, transformation temperatures, and the like, which can be selected to optimize the performance characteristics required. Nitinol is a commonly used shape memory alloy containing almost equal parts of titanium and nickel. Nitinol may, in certain embodiments, recover from significantly greater deformation compared to most other shape memory alloys.

The material may further comprise a temperature-sensitive shape memory material such that exposure of the connector to the heat of the subject's body may result in the connector being able to assume a second conformation different than a first conformation. For example, nitinol is also commonly used biomaterial with thermal shape memory properties. An inner member made from a temperature-sensitive shape memory alloy can be deformed (e.g., bent) to a shape suitable for insertion into an access member under conditions of limited clearance, with a thermally-induced reversal of the deformation (e.g., from bent to straight) when the connector element is threaded though the cannula. The applied heat can be from the surrounding tissue, or may be externally applied. Temperature-sensitive shape memory alloys are available in a wide range of transformation temperatures appropriate for the clinical setting, including those alloys (such as nitinol) that exhibit a transformation temperature at body temperature.

The methods and devices of the present invention may be particularly useful where the operator of the device (e.g., surgeon or physician) requires a bilateral access to utilize a tool for repair of the body part. For example, in one embodiment, the method of the present invention may comprise a method for sectioning at least a portion of a body part.

In an embodiment, the method may be used for percutaneous access and sectioning of a vertebral body. Or, the method may be used for percutaneous access and repair of an intervertebral disc. In certain embodiments, where the body part is a vertebral body, access may be extrapedicular or transpedicular.

The method may comprise the steps of: accessing the body part via a first entry point using a first access path and/or a first access member; accessing the body part via a second entry point using a second access path and/or second access member; and establishing a communication between the two access paths or access members. In an embodiment, the method may comprise positioning the first and second access members such that the distal ends of each access member are in position to communicate with each other, the two access members function in a coordinated manner. In certain embodiments, the step of establishing a communication between the access members may comprise: (i) passing a first inner member through the first access member; (ii) passing a second inner member through the second access member; and (iii) engaging the distal end of the first inner member with the distal end of the second inner member.

A variety of tools may be inserted into the access members to section the body part. In one embodiment, the first inner member comprises a saw. For example, the first inner member may comprise a first leader wire connected to a giggly saw; the leader wire may be positioned on the distal end of the giggly saw. Thus, in certain embodiments, the method may comprise the steps of pulling the distal end of the saw through the second access member, and alternately pulling on each end of the wire saw to cut through a portion of the body part positioned between the distal ends of the first and second access members.

Additionally or alternatively, in certain embodiments, a laser beam may be used for at least some of the sectioning. In one embodiment, a laser light is directed to the body part via the first and second access members in a manner so as to fracture a portion of the body part adjacent to the distal ends of the first and second access members.

Or, a liquid (e.g., dilute acid) may be used for at least some of the sectioning. Thus, in certain embodiments, a liquid is directed to the body part via the first and second access members in a manner so as to dissolve a portion of the body part adjacent to the distal ends of the first and second access members.

Once a portion of the body part has been cut away, it may be possible to use additional techniques to separate the two halves of the body part that is being sectioned. For example, in one embodiment, the method may further comprise inserting a balloon into the body part via at least one of the access members, and expanding the balloon to facilitate additional sectioning of the body part. For example, in one embodiment, kyphoplasty balloons, such as those commercially available from Kyphon Inc., may be used to facilitate further sectioning.

In some cases, the sectioned body part may need to be further repaired. For example, where the body part is a vertebral body, the vertebral body may be sectioned so as to insert a repair material in a portion of the vertebral body, to thereby correct for deformities in the shape of the vertebral body. However, the vertebral body wall may not be intact after the sectioning procedure. Thus, there may be a need to repair or temporarily seal any gaps or apertures in the vertebral body wall prior to emplacing a repair material (e.g., bone filler or cement) in the vertebral body. In one embodiment, an expandable device is used.

Thus, in some embodiments, the present invention comprises methods for inserting a device in a body part. In an embodiment, the body part is spinal tissue, such as an intervertebral disc or a vertebral body. In an embodiment, the method may comprise the step of inserting a device and a distal portion of an elongated member into a spinal tissue within a patient's body through a first percutaneous path, wherein the device is connected to the distal portion of the elongated member. The method may also comprise inserting a distal portion of a second elongated member into the spinal tissue through a second percutaneous path. In an embodiment, the elongated members comprise delivery members. The method may further comprise connecting the distal portion of the second elongated member to the device within the spinal tissue. Also, the method may comprise operating a proximal portion of the first elongated member and a proximal portion of the second elongated member to cooperatively control the device within the spinal tissue. In an embodiment, the distal end of the device comprises a portion that binds to the distal end of the member that is inserted into the vertebral body via the second percutaneous path.

In an embodiment, the present invention comprises a method of introducing a device into a spinal tissue. The spinal tissue may be an intervertebral disc. Or, the spinal tissue may be a vertebral body. The method may comprise: (a) establishing a first percutaneous access to a vertebral body in a subject; (b) establishing a second percutaneous access to the vertebral body; (c) passing at least a distal portion of a device into the vertebral body through the first percutaneous path; and (d) introducing at least a distal end of the device into the distal end of the second percutaneous path.

In one embodiment, the device comprises an expandable member. Alternatively, the device may comprise a cutting member. In an embodiment, the method may further comprising manipulating the spinal tissue with the device. For example, the spinal tissue may be cut, or expanded or filled with a structural support or bone repair material as described herein.

In one embodiment, the method may comprise alternating the device from a first configuration into a second configuration. Where the device is expandable, the first configuration may be an unexpanded configuration and the second configuration may be an expanded configuration. Where the device is a wire saw, the saw may be drawn back and forth within the spinal tissue. Thus, in an embodiment, an operator pulls on each end of the cutting tool in an alternating motion to cut through a portion of the body part that is positioned between the distal ends of the first and second access members.

The method may also comprise emplacing the device in the body part. Thus, in an embodiment, the method may further comprise disconnecting the device from the first elongated member, and disconnecting the device from the second elongated member. Also, the method may further comprising removing the first elongated member, and removing the second elongated member, while leaving the device implanted inside the spinal tissue.

Thus, in some embodiments, the method may further include inserting an expandable device, e.g., an expandable barrier, in the body part via at least one of the access paths and/or access members. Alternate embodiments of methods of inserting an expandable barrier are described herein. In one embodiment, the method may comprise passing a first inner member comprising an expandable barrier through a first access path. The expandable barrier may comprise distal and proximal elongated members attached to either end of the barrier. In an embodiment, the distal and proximal elongated members are used to deliver the barrier to the body part via an access path or access member as described herein.

The method may further comprise passing a second inner member through the second access path. In an embodiment, the access paths are established and/or maintained using access members. For example, in one embodiment, the access path comprises percutaneous access to the spine, and the access members are cannulas. Also, the method may comprise engaging the distal end of the first inner member comprising the distal elongated member of the expandable barrier with the distal end of the second inner member to position the barrier between the distal ends of the first and second access paths and/or the first and second access members. At this point, the barrier may be expanded. In one embodiment, the expandable barrier may comprise an inflatable membrane such that insertion of a liquid or a gas may be used to expand the expandable barrier.

In certain embodiments, the distal and proximal elongated members may be disconnected from the barrier and the barrier left in the body part. The access members, the second inner member, and the distal and proximal elongated members of the barrier first member may be removed at various points in the method (e.g., when the barrier is emplaced or before).

The access member used with the devices and methods of the present invention may provide a path to access a region or a body part that is located within a subject's body. The access member may be any type of device that can extend from the location of interest (e.g., a bone or an organ) to be accessible to a user of the access member. For example, the access member may be designed to extend from an internal body part in a subject to outside of the subject's body. The access member may comprise an elongated hollow member such as a hollow cylinder or a tube. Thus, in one embodiment, the tube may be designed to provide an access from outside of a living body to the internal body part. In one embodiment, the access members are substantially cylindrical in shape. For example, the access member may comprise a cannula, such as a cannula used to deliver a material to bone or another type of body part. One of ordinary skill in the art having the benefit of this disclosure would appreciate that the access members can be configured with other shapes and/or dimensions such as oval, hexagonal, octagonal, and the like.

In one embodiment, the access member may be configured to provide percutaneous surgical access to the predetermined location. As used herein, a percutaneous surgical access denotes passage through substantially unbroken skin, as for example, by needle puncture, a cannula or a catheter. In alternate embodiments, the percutaneous surgical access may comprises an incision ranging from about 0.1 to 4.0 centimeters (cm) in diameter, or from about 0.2 to 2.0 cm in diameter, or from about 0.25 to 1 cm in diameter. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter.

For example, in a typical percutaneous surgical repair of a spine, a cannula may establish a percutaneous path along its elongated axis to a vertebral body of one of the several vertebrae. The vertebral body extends on the anterior (i.e., front or chest) side of the vertebrae. The vertebral body comprises an exterior formed from compact cortical bone. Cortical bone is bone consisting of, or relating to, the cortex or outer layer of a bony structure. The cortical bone may enclose an interior volume of reticulated cancellous or spongy, bone (also called medullary bone or trabecular bone). Cancellous bone is bone having a porous structure comprising many small cavities or cells. The vertebral body is in the shape of an oval disc, and access to the interior volume of the vertebral body can be achieved, for example, by drilling an access portal through a rear (posterior) side of the vertebral body (a postero-lateral approach). The portal for the postero-lateral approach may enter at a posterior side of the vertebral body and extend anteriorly into the vertebral body. Alternatively, access into the interior volume of a vertebral body can be accomplished by drilling an access portal through one or both pedicles of the vertebra. This is known as a transpedicular approach. Or, the access into the interior volume of the vertebral body may be accomplished by drilling an access portal just anterior to one or both pedicles of the vertebra. This is known as a extrapedicular approach.

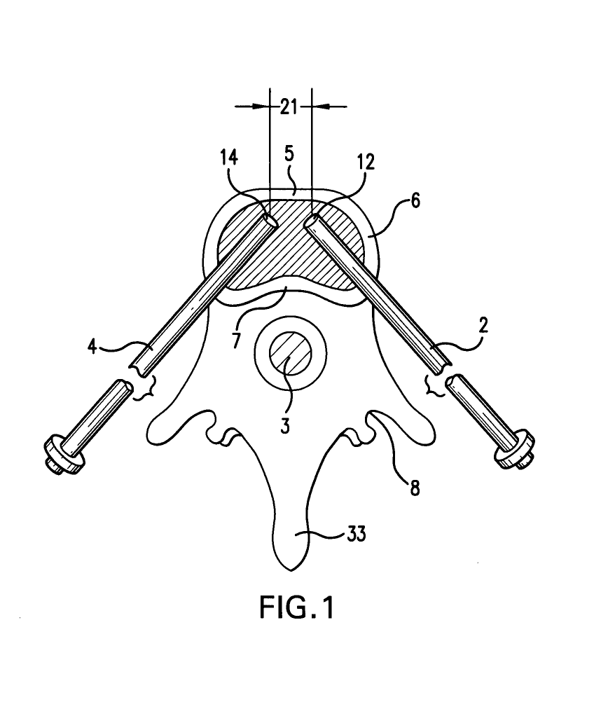

FIG. 1 shows one embodiment of device of the present invention being used to access a body part by a bilateral access. In one embodiment, the method may comprise inserting a first access member 2 and a second access member 4 into a body part 6. The body part shown in FIG. 1 is a vertebral body, however, this is by no means limiting with respect to the possible uses for the methods, devices and systems or kits of the present invention. Also shown in FIG. 1 is the pedicle bone 8 of the vertebral body and the spinal cord 3. The access members may comprise a hollow cylinder. For example, in one embodiment, the access members may comprise a cannula. In one embodiment, a cannula such as the catheters used to access and repair a bone may be used.

The access as shown in FIG. 1 comprises an extrapedicular access (i.e., outside of the pedicle bone). Other types of access (e.g., transpedicular) may be provided depending upon the procedure being performed. Thus, it can be seen that in one embodiment, the bilateral extrapedicular access provides a means to access the interior of the vertebral body without having to section through the pedicular bone, and without endangering the spinal cord.

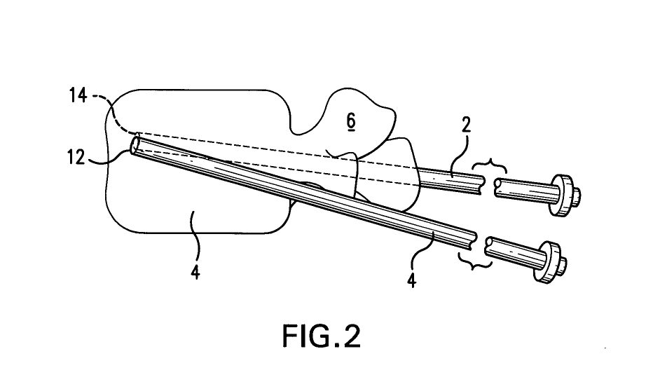

FIG. 2 shows a side view of the bilateral access shown in FIG. 1. It can be seen from FIGS. 1 and 2 that the distal ends 12, 14 of access members 2 and 4 may be positioned in close proximity to each other. By close proximity, it may be understood that the distance 21 between the ends 12, 14 of the access members (FIG. 1) is such that the two access members may be manipulated in a coordinated fashion to repair or modify the body part into which they are inserted. In one embodiment, the distance 21 between the ends 12, 14 of the two access members is such that an item may be passed between one access member into the other access member. In this way, the two access members may be considered to be in communication with each other. In alternate embodiments, the distance between the distal ends of the first and second access members may range from about 0.1 mm to 5 cm, or from about 1 mm to about 2 cm, or from about 2 mm to about 0.5 cm, or from about 4 mm to 8 mm apart. Or, ranges within these ranges may be used.

In one embodiment, imaging techniques may be used to facilitate positioning of the access members. Such imaging techniques may include fluoroscopy, CT scanning, X-ray imaging and other techniques known in the art. For example, in one embodiment, the two ends of the access members will be juxtaposed close to the anterior cortical wall. When seen from a lateral (side) view, the ends of the access members should have the distal ends superimposed.

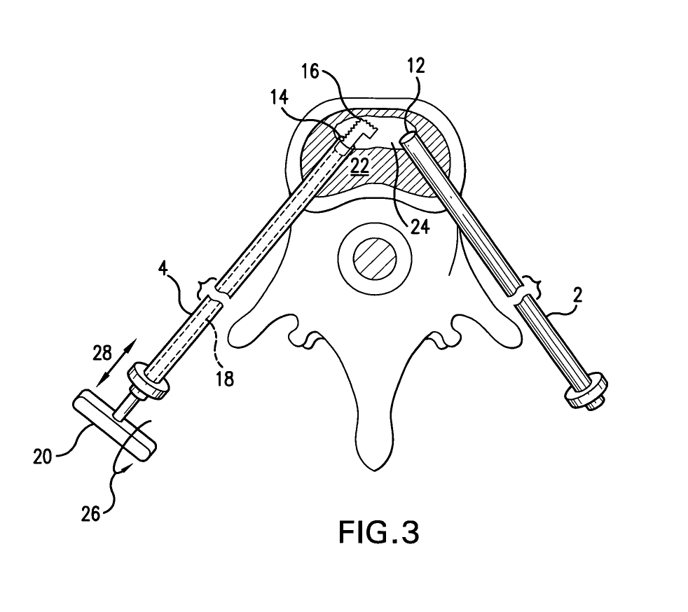

FIG. 3 shows a top view of a vertebral body accessed by a first access member 2 and a second access member 4. Also shown is a curette blade 16 attached to a stylet or rod 18. The stylet (rod) may comprise a handle 20 that allows the curette to be manipulated to thereby cut away some of the interior of the body part. For example, where the device is used to access a vertebral body, the curette can be used to cut away some of the cancellous bone 22 in the interior of the vertebral body to provide an opening or path 24 between the distal ends 12, 14 of the two access members 2, 4. Thus, the handle may be rotated 26, or pushed and pulled 28 to facilitate cutting the interior of the body part.

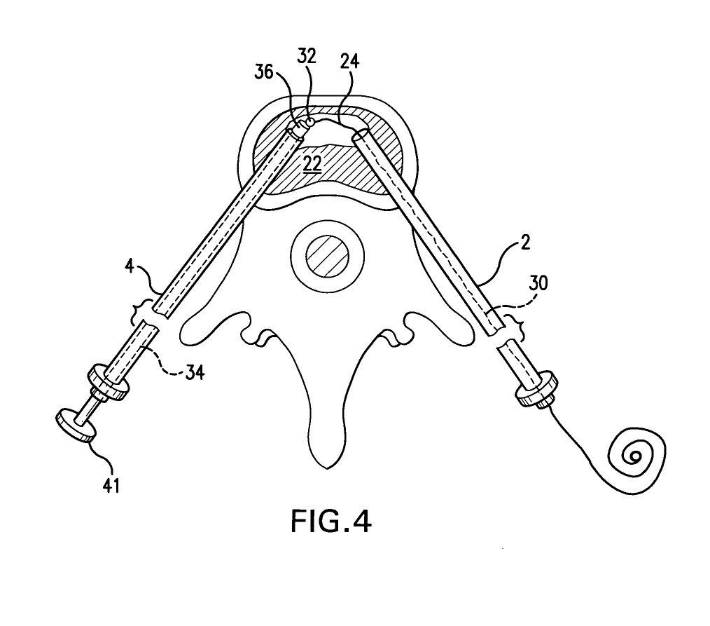

Once a bilateral access has been established, and a path 24 has been made to allow for communication between the two access members 2, 4, the method may comprise the step providing a tool into the body part via the access members. As shown in FIG. 4, in one embodiment, a first inner member 30 is inserted into the first access member 2. The first inner member may comprise a tool that can be used to modify the structure of the body part. For example, the first inner member may comprise a wire saw. Or, the first inner member may comprise an expandable barrier as discussed in more detail below. Or, other tools may be inserted as the first inner member.

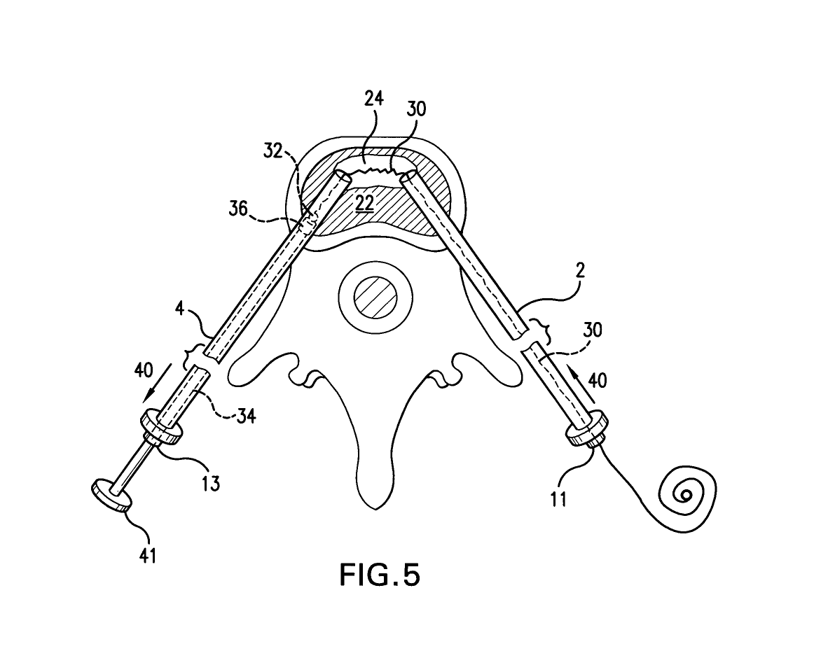

Referring now to FIGS. 4 and 5, the method may comprise the step of engaging the first inner member 30 with a second inner member 34 that is inserted into the second access member 4 (FIG. 4), and then using the second inner member to pull the first inner member through the first access member 2 and into the second access member 4 (FIG. 5). In this way, a connection or communication is established between the two access members. In certain embodiments, the second inner member 34 may also comprise a rod or a stylet. For example, the rod or stylet used to curette a path between the two access members may be used.

The inner members 30, 34 may comprise distal ends that are able to engage each other. For example, the second inner member 34 may comprise a rare earth magnet 36. The first inner member 30 may comprise a fixture 32 at its end that is attracted to the magnet 36 such that when the two ends 32, 36 of the two inner members are juxtaposed, they will be connected to each other. By pulling on the proximal end (e.g. handle 41) of the second inner member 34, the first inner member 30 will be pulled through the first access member 2 and into the second access member 4 (FIG. 5).

In one embodiment, the means for connecting the two inner members comprises a magnet and a material that is attracted to, and thus, binds to the magnet. The magnet may comprise a rare earth magnetic material such as neodymium, neodymium-iron-boron, samarium-cobalt 1-5, or samarium-cobalt 2-17. The material attracted to the magnet may comprise a high carbon content steel, chrome steel (AISI E-52100), hardened stainless steel (AISI Type 440C), soft mild steel (AISI Type 1018), or a magnetic or rare earth magnetic material as listed above.

Or, other materials known to bind to each other by means of a physical or chemical bonding may be used. For example, in alternate embodiments, a chemical bond could be achieved using adhesive-coated distal ends. In one embodiment, the distal end of one inner member coated with a polymeric material may come into contact with the distal end of the second inner member, where the distal end of the second inner member is made from a compound that can polymerize with the polymeric material on the first inner member. For example, a first inner member having a polyurethane coating may be linked to a second inner member also coated with polyurethane by including an initiating agent (e.g., chemical catalyst or a UV light). Or, a unsaturated compound present at the end of the inner members may be polymerized by including an initiating agent (e.g., chemical catalyst or a UV light) to promote polymerization. Or, other embodiments for forming chemical bonds may be used.

Or a physical connector, such as a hook and loop system may be used. Or, the distal end of one inner member may comprise a protrusion that fits within an aperture or well on the end of the other inner member. Or, in one embodiment, one of the inner members may comprise a micro-rongeur that is able to couple the distal end of the other inner member. Or, a vacuum or suction device may be used to attach to a ball or other shape when the end of one member comes into contact with the distal end of a second member

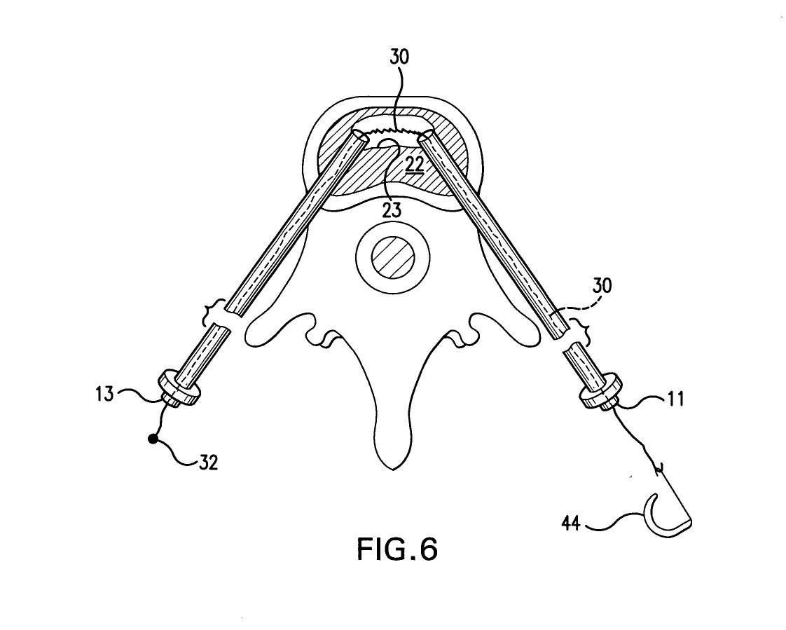

By pulling 40 the second inner member 34 through the second access member 4 (i.e., towards the proximal end of the second access member) (FIG. 5), the first inner member 30 may be pulled completely through the second access member. FIG. 6 shows a point in the procedure where the first inner member has been pulled through the proximal end 13 of the second access member 4.

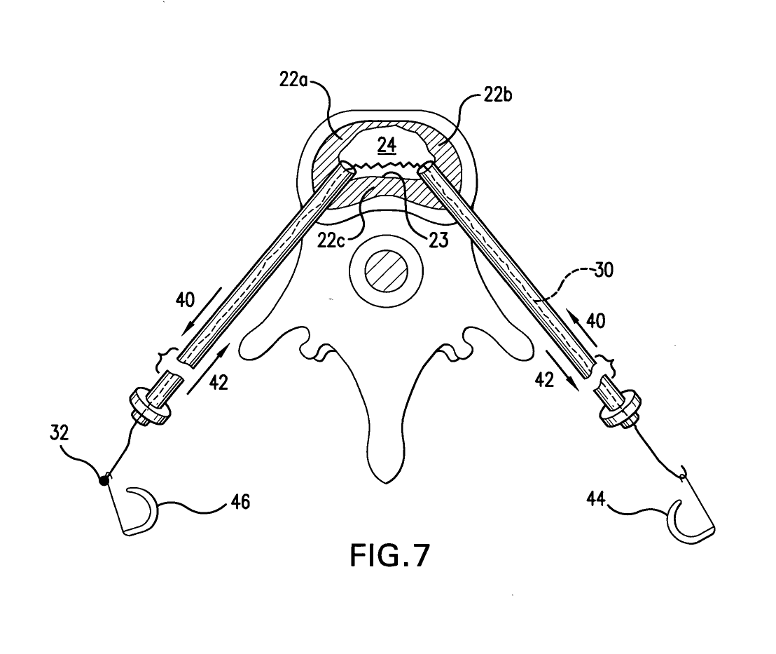

At this point, a user, such as a physician or other health care professional, may grasp the ends of the first inner member to manipulate the tool. In one embodiment, the ends of the first inner member may comprise handles 44, 46 or other means to grip the ends of the inner member. In one embodiment, at least one of the handles is attached to the first inner member after the first inner member has been pulled through the two access members (FIG. 7). Or, the handles may comprise a collapsible loop that can function as the distal ends of the first inner member. Such a loop may have a ferrule fixture attached to the end of the loop.

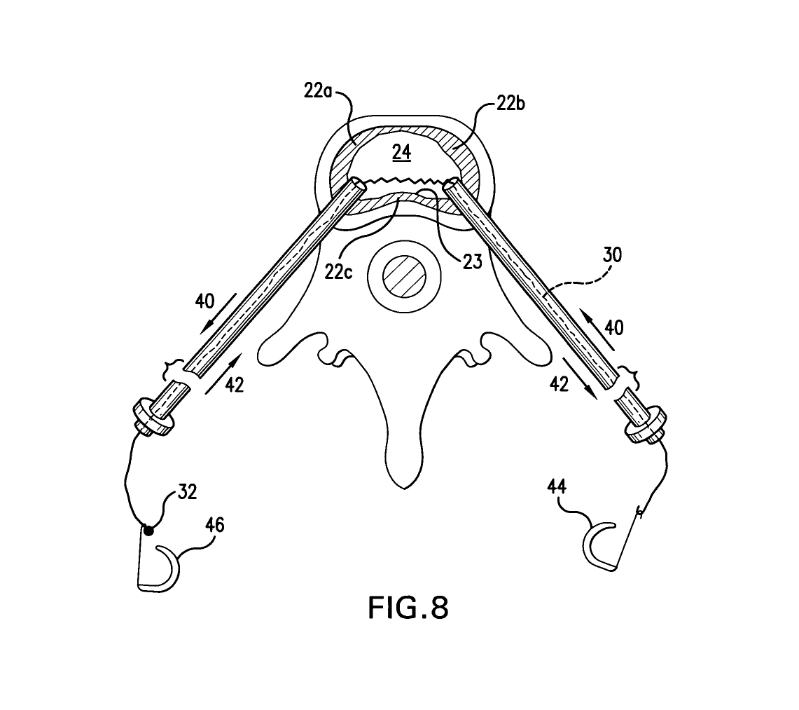

By pulling the ends of the inner member 30 in alternating directions 40, 42 the inner member may move across the face of the interior of the body part being accessed. Where the inner member 30 is a saw, the inner member may be juxtaposed against the bone in the interior of the vertebral body to slice away at the surface 23 of the bone 22. Thus, as shown in FIGS. 6, 7 and 8 the interior of the vertebral body may be sawed through to provide an increasingly larger void space 24, moving in a direction from anterior to posterior. In one embodiment, the void space is bounded by anterior-lateral cancellous bone 22a, 22b that was positioned exterior to the two access members 2, 4 and so was not cut away by the saw 30, and posterior cancellous bone 22c, comprising cancellous bone that has not yet been cut by the saw.

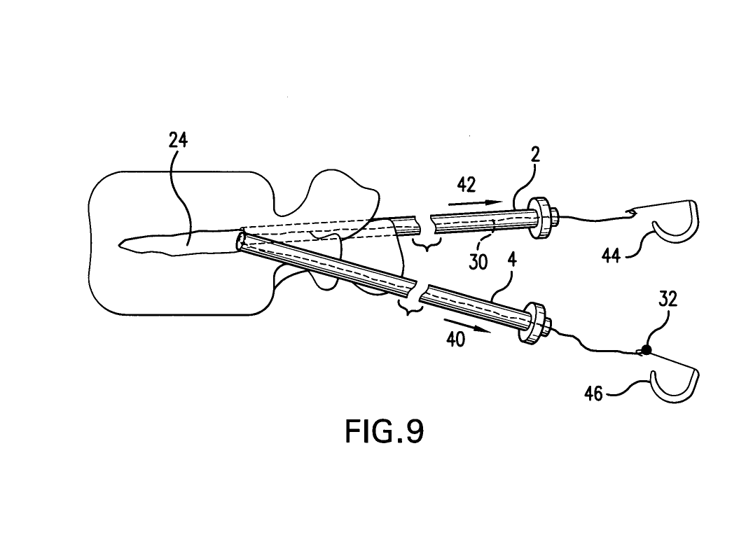

In one embodiment, the progress of the bone removal is monitored by fluoroscopy or other imaging techniques. In this way, the cutting does not progress so far as to endanger the spinal cord. FIG. 9 shows a side view of a vertebral body that has been sliced using a bilateral cannula access of the present invention, and illustrates a point at which the sectioning of a vertebral body may be terminated.

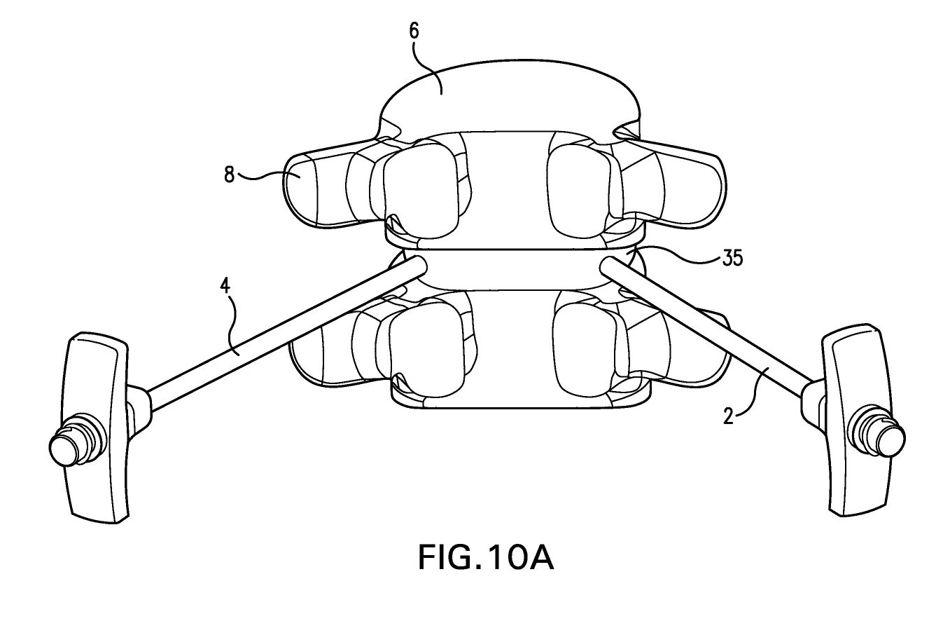

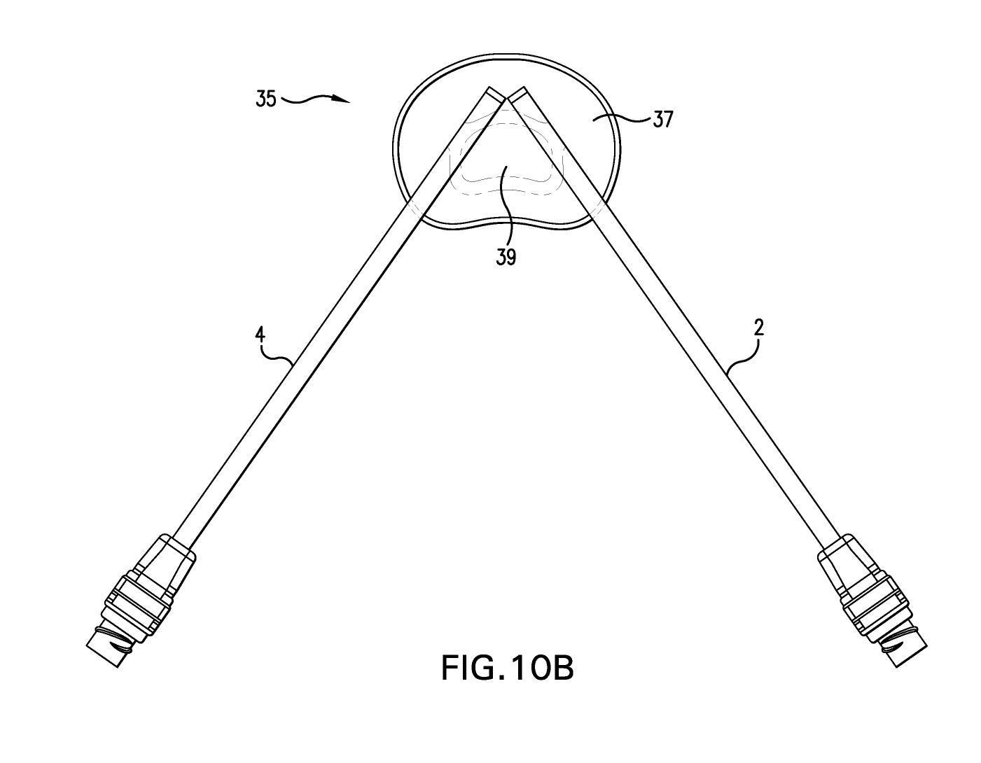

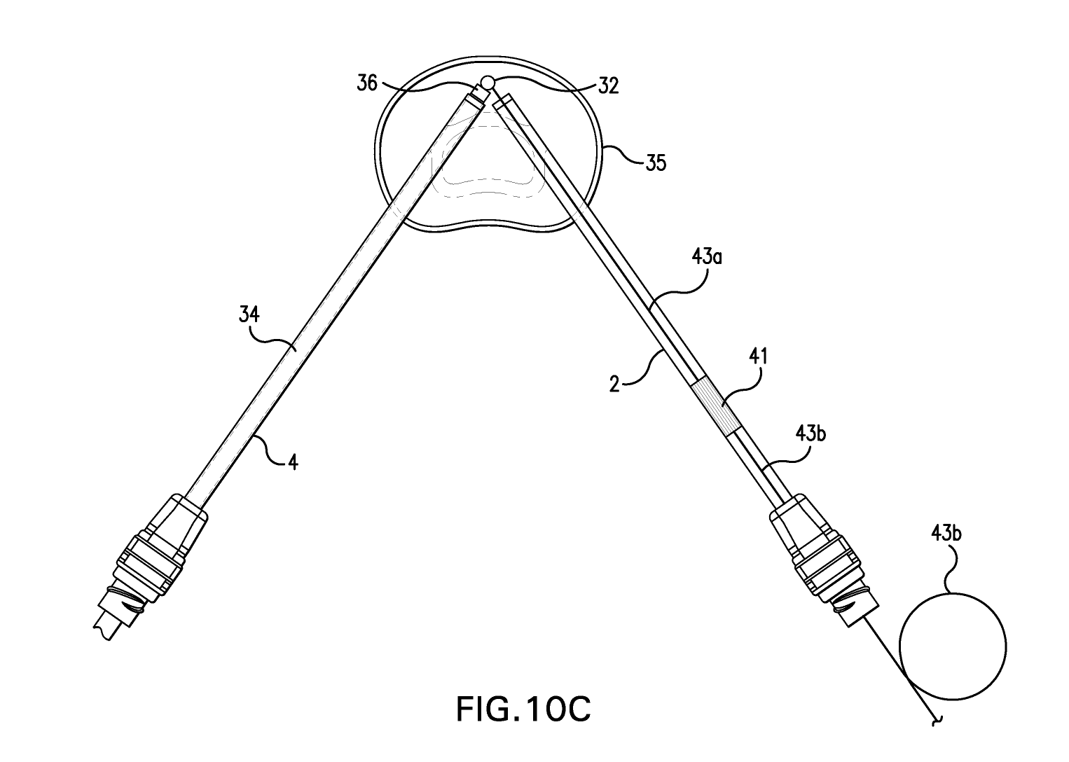

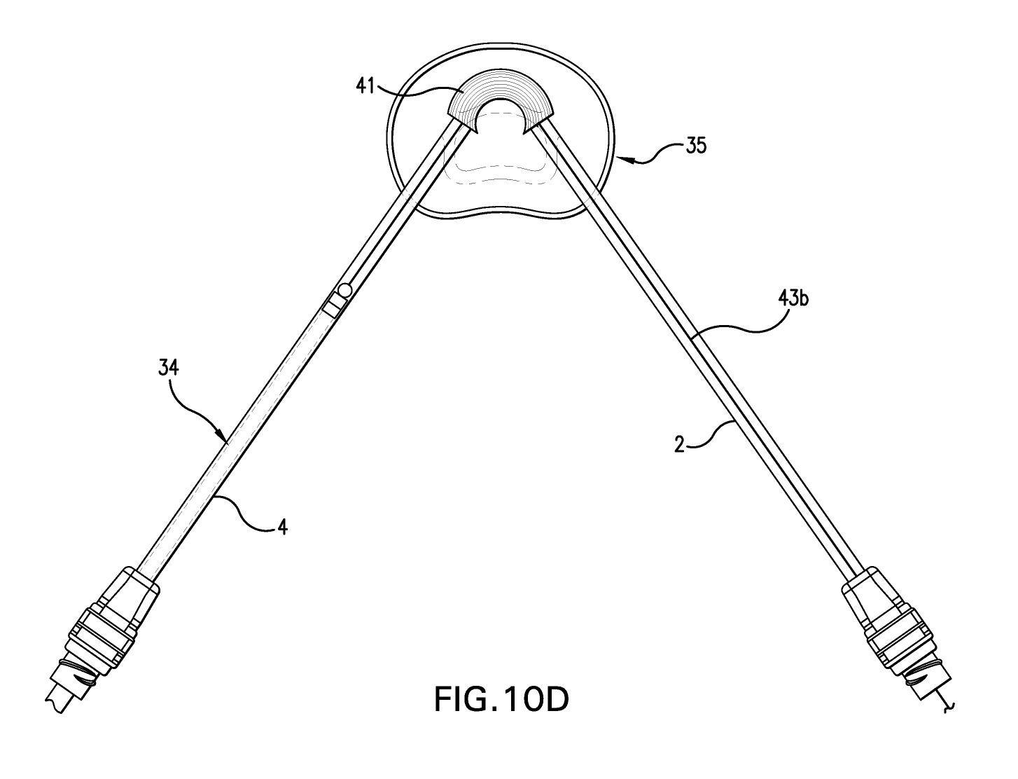

As described herein, in certain embodiments, the access members may be emplaced in an intervertebral disc. FIG. 10 shows an example, of two access members inserted into an intervertebral disc 35 using a bilateral approach. Thus, as shown in FIG. 10A, two cannulas 2,4 may be inserted into the disc using an entry angle that parallels the pedicular bone 8 of the vertebral body 6. The cannulas may be inserted into the annulus 37 until the meet, close to the anterior wall. In this way, the cannulas may straddle nucleus 39 of the disc (FIG. 10B). Next, a second inner member 34, such as a stylet having a magnet 36 on the end may be inserted into one of the cannulas 4, and a first inner member tool 41 having a distal elongated member 43a with a ferrule tip 32 and a proximal elongated member 43b is inserted into the other cannula 2. The second inner member stylet and the second inner member tool may be advanced through the cannulas until the magnet is able to bond to the ferrule tip (FIG. 10C). The second inner member stylet 34 may then be used to pull the first inner member tool 41 through the second cannula 4 via the distal elongated member 43a that interacts via the ferrule tip 32 with the magnet 36 (FIG. 10D). Once the tool 41 is in place, it may be manipulated by an operator via the end 43a and 43b of the tool, or using handles attached to the ends of the tool.

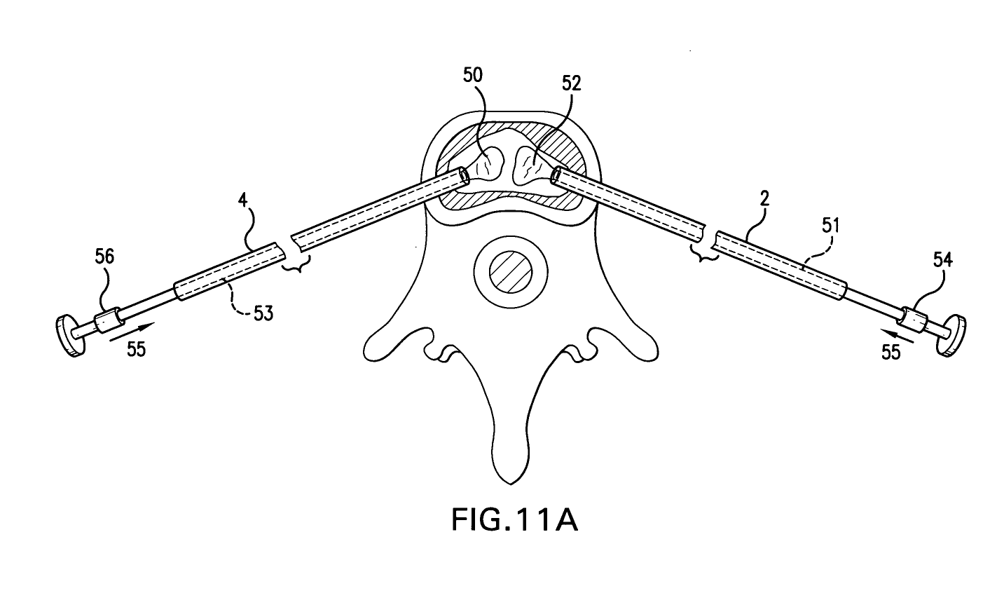

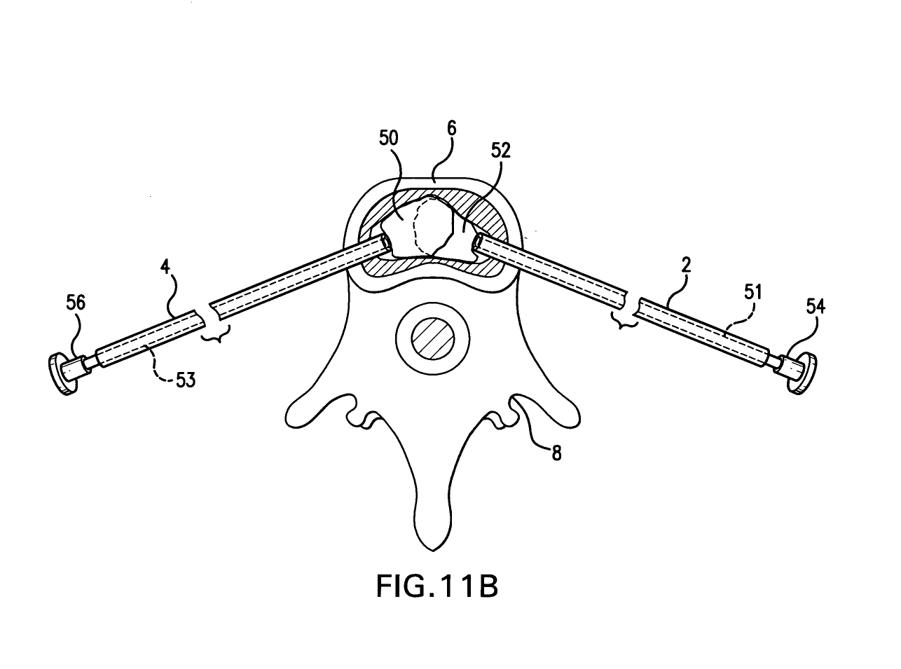

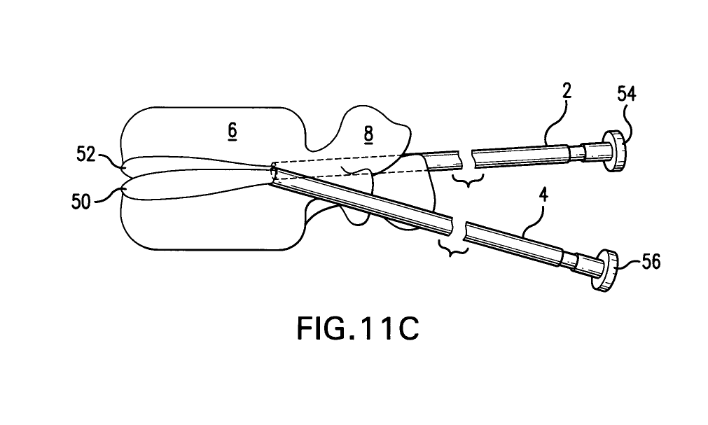

In some cases, additional tools may be inserted into the body part as needed. For example, in one embodiment, inflatable members (e.g., balloons) are inserted into the body part to facilitate sectioning of the body part. FIG. 11A shows balloons 50, 52 inserted via the two access members 2, 4 to separate the partially sectioned vertebral body into a superior and an inferior portion. In one embodiment, the balloons are inflated (FIGS. 11B and 11C) using inner cannulas 51, 53 by pushing 55 a gas or liquid into the inflatable membranes using plungers 54, 56 as is known in the art.

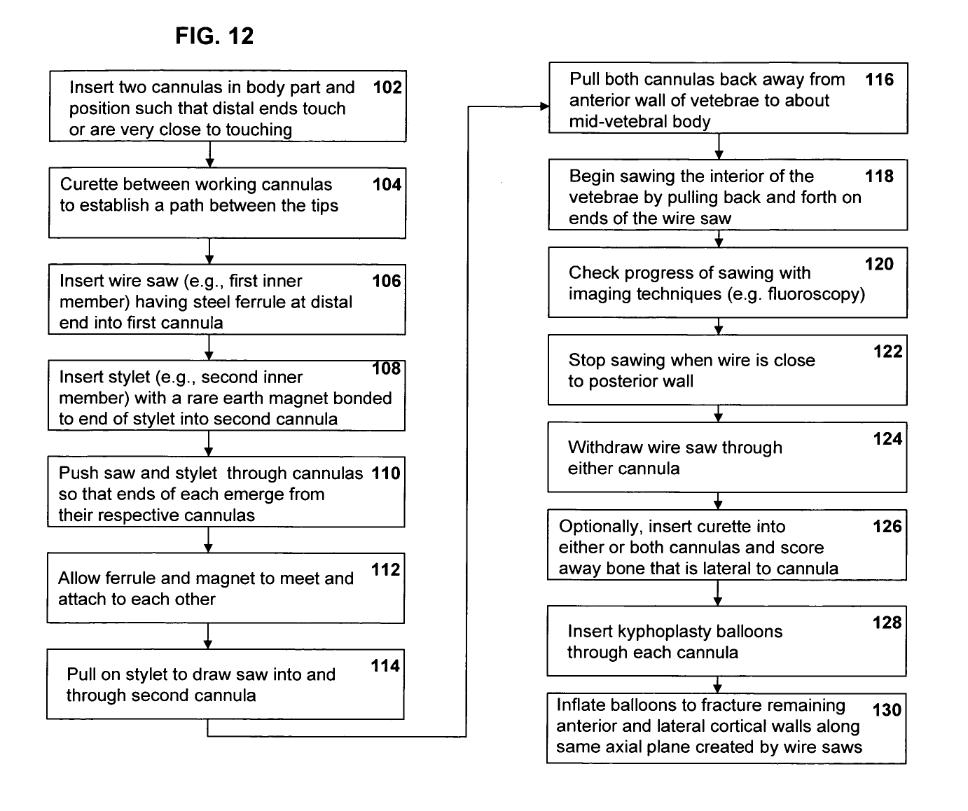

FIG. 12 provides an overview of a method of using a multipoint access device to access a body part. Although the example shown as FIG. 12 is the use of a multipoint delivery device to section a vertebral body, the methods and devices may be used for other procedures known in the art of bone repair. For example, a multipoint delivery device of the present invention may be used to deliver and emplace an expandable barrier as described below. Or, a multipoint delivery device of the present invention may be used to deliver and emplace a bone repair material where a single access is not able to reach the portion of the body part requiring repair. Or, a multipoint delivery device of the present invention may be used to introduce a laser beam into a body part as needed for repair of the body part. In yet another embodiment, a multipoint delivery device of the present invention may be used to introduce a liquid that can be used to section a bone, such as a dilute acid solution. Or, a multipoint delivery device for performing a fusion or corpectomy of the vertebral body may be used.

Thus, as shown in FIG. 12, in one embodiment, two cannulas may be used for extrapedicular approach to access a vertebral body. The cannulas may be inserted in such a manner so as to meet at the medial plane close to the anterior cortical wall. In one embodiment, the working cannulas are superimposed in the lateral view, or at least have their tips laterally superimposed 102. Still referring to FIG. 12, at this point, the operator may insert a stylet having a curette at one end into one of the cannulas and push the stylet through the cannula such that the blade emerges from the distal end of the cannula. The curette blade may be used to clear a path between the distal ends of the two cannulas 104.

Next, a wire saw may be inserted into the end of one of the cannulas and threaded through the cannula such that the end of the saw begins to emerge from the distal end of the cannula. The distal end of the wire saw may have a ferrule piece bonded to the tip 106. Also, a stylet having a magnet, such as a rare earth magnet, may be inserted into the second cannula and threaded through the cannula such that the end of the stylet begins to emerge from the distal end of the cannula (108). When both the stylet and the wire saw emerge from the distal ends of their respective cannulas (110), the magnet will draw the ferrule towards it, thereby linking the saw to the stylet (112). In this way, a communication is established between the two cannulas. At this point, the wire saw may be drawn up through the second cannula by pulling the stylet out of the proximal end of the second cannula (114).

Once the wire saw has been pulled through the cannulas, the operator may pull the two cannulas slightly towards the posterior of the spine (i.e., away from the anterior wall). In one embodiment, the cannulas are pulled back such that the distal ends of the cannulas are positioned at about the middle of the vertebral body (116). The operator may then begin cutting through the cancellous bone by pulling back and forth on the saw, and thereby drawing the saw across the bone positioned posterior to the path previously cut by the curette (118). The operator may cut through the vertebral body moving from anterior to posterior and using the ends of the cannulas to restrict the posterior progress of the saw as needed.

The progress of the cutting may be monitored by an imaging technique (120). For example, fluoroscopy may be used to monitor the progress of the saw. In one embodiment, a lateral view may provide a determination of the progress of the cutting. Thus, the method may comprise a series of steps whereby the operator: (a) pulls back on the cannulas; (b) cuts away additional material from the body part; and (c) checks the progress by fluoroscopy. This series of steps ((a), (b) and (c)) may be repeated until the wire saw is at or near the posterior wall.

Once the wire saw is at, or near, the posterior wall (122), the sawing operation may be terminated and the saw may be removed by pulling it out of one of the cannulas (124). The interior region of the vertebral body that has been sectioned by the wire saw may then comprise a triangular area that is defined by the borders of the bone abutting the posterior vertebral wall (see FIG. 7: 22c) and the bone exterior to the two cannulas as positioned within the vertebral body (See FIG. 7: 22a, 22b).

At this point, the stylet having a curette may be inserted back into the void in the body part via either cannula (126). The curette may be used to score away bone that remains in the body part (e.g., bone that was lateral to the outside of the cannula). The curetting may help enlarge the sectioned area of the vertebral body to thereby facilitate further sectioning of the vertebral body.

Next, a device may be inserted into the cannulas to facilitate fracturing the remainder of the bone that was not cut away by the saw or curette. In one embodiment, inflatable members (e.g., kyphoplasty balloons) may be inserted into each cannula (128). The balloons may then be simultaneously inflated to thereby fracture additional bone in the plane of the excised material (e.g., the cancellous bone that is anterior to the area that has been sawed through or sectioned with the saw). Thus, in one embodiment, the lifting force of the balloons will serve to fracture at least a portion of the remaining anterior and lateral cortical walls along the same axial plane that was created by the wire saw. Using two balloons can help to spread the lifting force over two areas thus facilitating fracture of the remaining bone. When the walls are fractured, the sectioning of the vertebral body into a superior (upper) and an inferior (lower) portion will be complete.

Other tools that may be inserted in this manner include tools that can be used to perform fusions and corpectomies. With regards to fusion, a tool capable of disrupting the nucleus (center of an intervertebral disc) and the annulus (fibrous outer border of the intervertebral disc), such as a continuous abrasive wire brush, may be inserted using a bilateral access (e.g., two cannulas via a percutaneous approach), and pulled across the interior of the disc. Pulling an abrasive tool across the interior of the disc may disrupt the interior disc material. Also, in certain embodiments, the tool may be designed to entrap the disrupted material within the wire mesh, thereby progressively reducing the material within the disc in preparation for fusion. Once sufficient disc material is removed, a tool, such as an abrasive wire, may be inserted into the disc space, again using the bilaterally emplaced access members, to prepare the endplates for fusion. Once the second tool (e.g., abrasive wire) is inserted, the tool may be used to scrape any remaining disc material off of the endplates, to ensure appropriate conditions for proper fusion. The abrasive wire may be scraped across the endplates in various directions to accomplish this task. Finally, a collapsible fusion cage, similar to a stent-like device that can be expanded once it is in place, may be inserted into the interbody disc space using this method. Once in place, the structure may be actuated, thereby expanding the structure to take up the space once occupied by the removed disc material. The fusion cage may be expanded/deployed in the disc space using coordinated movements through the first and second access members. Once deployed, the expanded cage can be packed with bone graft through either or both of the access members.

Similarly, a bilateral approach may be used for corpectomies of the vertebral body, inserting tools to clear away debris and degraded material from the interior of the vertebral body, followed by inserting an expandable device (e.g., expandable cage) to support the vertebral body. Again, once deployed, the expanded cage can be packed with bone graft through either or both of the access members. Thus, for a corpectomy of a vertebral body, in an embodiment, an abrasive or continuous wire brush tool may be inserted into the interior of a vertebral body using bilateral access path or bilaterally emplaced access members. In an embodiment, an abrasive tool (e.g., wire brush) can be pulled across the interior of the vertebral body, thereby aggressively eroding away any degraded material and simultaneously removing the material as the disrupted material becomes entrapped within the wire mesh. Once a sufficient amount of material has been removed from the interior of the vertebral body, an endplate preparation structure (e.g., an wire cage designed to grip the endplate) may be inserted into the vertebral body using the bilateral access paths (e.g., bilaterally emplaced access member cannulas). In an embodiment, a wire structure may be scraped across the endplates (of the superior and inferior vertebral bodies) to remove any remaining material, and to ensure proper fusion. Finally, a collapsible corpectomy cage, similar to a stent-like device that can be expanded once it is in place, can be inserted into the vertebral body space using this method. Once in place, the structure may be actuated to expand the structure to take up the space once occupied by the removed vertebral body and adjacent discs. The corpectomy cage may be expanded/deployed in the vertebral body space using coordinated movements through the first and second access members. One deployed, the expanded cage could be packed with bone graft through either or both of the access members.

### Devices for Multipoint Emplacement

Other embodiments of the present invention may comprise devices for emplacing in a body part and methods of emplacing such devices. In one embodiment, the device is expandable. In some embodiments, the device may be emplaced in a bone. In one embodiment, the bone may comprise a vertebral body. For example, the device may be emplaced in a vertebral body that has been fractured using a vertebral sectioner. Or, the device may be emplaced in a bone that has been damaged or has a section that has been broken off and that needs to be repaired.

In some embodiments, the present invention comprises methods for inserting a device in a body part. In an embodiment, the body part is spinal tissue, such as an intervertebral disc or a vertebral body. In an embodiment, the method may comprise the step of inserting a device and a distal portion of an elongated member into a spinal tissue within a patient's body through a first percutaneous path, wherein the device is connected to the distal portion of the elongated member. The method may also comprise inserting a distal portion of a second elongated member into the spinal tissue through a second percutaneous path. In an embodiment, the elongated members comprise delivery members. The method may further comprise connecting the distal portion of the second elongated member to the device within the spinal tissue. Also, the method may comprise operating a proximal portion of the first elongated member and a proximal portion of the second elongated member to cooperatively control the device within the spinal tissue.

In an embodiment, the device comprises an expandable member. Alternatively, the device may comprise a cutting member. In an embodiment, the method may further comprising manipulating the spinal tissue with the device. For example, the spinal tissue may be cut, or expanded or filled with a structural support or bone repair material as described herein.

For example, in an embodiment, the method may comprise alternating the device from a first configuration into a second configuration. Where the device is expandable, the first configuration may be an unexpanded configuration and the second configuration may be an expanded configuration.

The method may comprise emplacing the device in the body part. Thus, in an embodiment, the method may further comprise disconnecting the device from the first elongated member, and disconnecting the device from the second elongated member. Also, the method may further comprising removing the first elongated member, and removing the second elongated member, while leaving the device implanted inside the spinal tissue.

In some embodiments, the device comprises an expandable device. For example, the device may comprise an expandable barrier. Thus, the present invention may comprise methods for emplacing an expandable barrier in a body part. The expandable barriers of the present invention may be used to prevent leakage of a repair material (e.g., bone cement) during the repair of the body part.

Thus, in certain embodiments, the present invention may comprise an expandable device for emplacement in a body part. In one embodiment, the expandable device may comprise an expandable portion comprising a surface having a first unexpanded shape and a second expanded shape. Also, the expandable device may comprise a delivery member for delivering the expandable portion to a body part.

In one embodiment, the expandable device may be designed to be accessed by an operator at two different places on the device. For example, in some embodiments, the expandable device may comprise an expandable portion having a first end and a second end, and a first delivery member for delivering the expandable portion to a body part, wherein the first delivery member comprises a first part attached to the one end of the expandable portion and a second part attached to the second end of the expandable portion.

The expandable device may comprise a means to deliver the expandable device to a body part. Thus, in one embodiment, the expandable device may comprise a delivery system, where the delivery system comprises a first access member and a second access member. The delivery system may also comprise a second delivery member to be inserted into the second access member. In one embodiment, the distal end of the first delivery member attached to the expandable portion comprises a first binding component that can engage and bind to a second binding component on the distal end of the second delivery member.

Other embodiments of the present invention comprise methods for emplacing an expandable device in a body part. In one embodiment, the expandable device may be used to seal the body part. For example, the expandable device may be used to seal an aperture in the body part. The methods may be embodied in a variety of ways.

In one embodiment, the method for emplacing an expandable device in a body part may comprise: (a) emplacing the expandable device into the body part via at least a first access member; (b) positioning the expandable device within the body part via the first access member and a second access member that is in communication with the first access member; and (c) expanding the expandable device. The method may further comprise positioning the expandable device over an aperture and expanding the expandable device so as to seal the aperture. The method may be used on a variety of body parts. In one embodiment, the body part comprises a vertebral body. Alternatively, the body part may comprise an intervertebral disc.

In certain embodiments, the method comprises a multipoint access of the body part. For example, in some cases a multipoint access may facilitate emplacing an expandable device in a body structure, where it is difficult to emplace the device using a single access member.

For example, in some embodiments, the expandable device may be implanted using a bilateral access to the body part of interest. Thus, in one embodiment, the method may comprise the step of positioning the expandable device at an aperture in a body part by urging the expandable device into the body part via two access members that are in communication with each other. Also, the method may comprise expanding the expandable device to seal the aperture. In certain embodiments, the step of positioning the expandable membrane in the body part may comprise manipulating both ends of the expandable member. Thus, the method of emplacing the expandable device in a body part may, in certain embodiments, comprise: (i) accessing the body part via a first entry point using a first access member; (ii) accessing the body part via a second entry point using a second access member; (iii) positioning the first and second access members such that the distal ends of each access member are in position to communicate with each other; and (iv) establishing a communication between the two access members, such that the two access members function in a coordinated manner to emplace the expandable device.

In one embodiment, the access members function as part of a delivery system that may be used to deliver and position the expandable portion of the device in a body part. For example, in certain embodiments, the expandable device comprises a first delivery member for delivering the expandable portion to a body part, wherein the first delivery member comprises a first part attached to the one end of the expandable portion and a second part attached to the second end of the expandable portion. The delivery system may further comprise a second delivery member that interacts with the first delivery member to position the expandable device in the body part. Thus, the delivery system may comprise a means to have the first and second delivery members interact, to thereby form a communication between the first and second delivery members such that the expandable member can be positioned in the body part of interest. In one embodiment, the method comprises passing a second delivery member through the second access member; and engaging the distal end of the first delivery member with the distal end of the second delivery member such that the distal end of the first delivery member engages the distal end of the second delivery member.

A variety of methods may be used to establish a communication between the two delivery members. For example, in one embodiment, the distal end of the second delivery member comprises a rare earth magnet, and the distal end of the first delivery member attached to the expandable portion comprises a material that binds to the rare earth magnet. For example, the distal end of the first delivery member may comprise a ferrous material. Or, in one embodiment, the distal surface of the first delivery member may comprise a magnet and the distal surface of the second delivery member may comprise a material that binds the magnet.

In other embodiments, a fastening device may be used to engage the two delivery members. For example, the distal end of one delivery member may comprise a hook and the distal end of the other delivery member may comprise a loop. Or, the distal end of one delivery member may comprise a protrusion (e.g., a male fitting) that fits within an aperture or well (e.g., a female fitting) on the end of the other delivery member. Or, in one embodiment, one of the delivery members may comprise a micro-rongeur that is able to couple the distal end of the other delivery member.

Another embodiment may have the second inner member take the form of a collapsible claw or basket. This claw can be made from aluminum, stainless steel, spring steel, nickel titanium alloys, or other alloys. Or, in some embodiments, the claw may be made of plastic. For example, a resilient plastic such as vinyl, nylon, polypropylene, a polyethylene, ionomer, polyurethane, and polyethylene tetraphthalate (PET) may be used. The first inner member may be comprised of a flexible, string-like member and have a ferrule, possibly spherical in shape, bonded to its distal end.

For example, where a claw and ferrule are used, both the first and second inner members may be advanced down their respective access members such that ferrule on the distal end of the first inner member would lie within or pass through the jaws of the claw of the second inner member. Once the inner members are in this position, the basket may be collapsed around the distal end of the first inner member. For example, a sheath may be translated over the second inner member thereby collapsing the claw around the ferrule. Or, the basket may comprise an actuating mechanism, such as a wire, that when pulled by an operator, collapses the basket. With the ferrule and accompanying first inner member captured within the claw of the second inner member, the second inner member may be pulled back through the second access member by pulling the first inner member up and through the second access member.

Or, chemical bonding methods may be used as described herein.

There may be a variety of methods whereby the expandable device may be expanded. In one embodiment, the expandable portion may comprise a structural member that urges the expandable portion from a first unexpanded shape to a second expanded shape. To expand the expandable portion, the structural member is attached to at least a portion of the expandable portion. In one embodiment, the structural member comprises a framework. In one embodiment, the framework may comprise a single wire. In other embodiments, the framework may comprise a plurality of wires.

The expandable portion may be made of any material that can be functionally expanded in the body part of interest. For example, the expandable portion may comprise a membrane. Alternatively or additionally, the expandable device may comprise an inflatable membrane. Thus, in certain embodiments, the expandable device may be expanded by the introduction of a gas or fluid into the expandable membrane. Membranes that may be used include PET, Nylon, Poly(lactic acid) (PLA), Poly(glycolic acid) (PGA), Polydioxanone (PDS) or a copolymer.

In one embodiment, the expandable device may comprise a biodegradable membrane. Thus, in one embodiment, the membrane may be seal a portion of a body part that is exposed to the exterior. Once the membrane is positioned, a repair material may be inserted into the body part. As the material hardens, it will be positioned adjacent to the membrane. If the membrane is biodegradable, it may decompose in situ over time, thereby leaving the deposited repair material to repair and seal the body part. Alternatively, where an inflatable, biodegradable membrane is used, the membrane may be filled with a repair material needed to be emplaced in the body part. Again, with time, the biodegradable membrane surrounding the repair material may decompose, thereby leaving the repair material emplaced in the body part. If the membrane is relatively thin, the loss of volume upon degradation of the membrane may be inconsequential.

In yet another embodiment, a multipiece barrier may be used. For example, the barrier may comprise two separate portions that interlock, such that the distal end of a first portion comprises a surface is at least partially on top of a surface of the distal end of a second portion where the two halves of the barrier meet. In an embodiment, the two portions comprise fixtures that can interlock. For example, one half of the barrier may comprise a male fixture and the other portion of the barrier may comprise a female fixture. In yet another embodiment, the multipiece barrier may have two pieces that meet at their distal ends and become attached to each other. In certain embodiments where a multipiece barrier is used, the two portions of the barrier may each be attached at their proximal ends to an elongated member that is used to deliver the parts of the barrier to the body part.

The delivery members may comprise an elongated member that is attached to the device. The delivery member(s) used to emplace an expandable member may comprise a material that is flexible, such that the delivery member(s) are able to bend. Alternatively, the delivery members may be relatively rigid. Also, in one embodiment, the delivery member(s) (e.g., the first delivery member attached to the expandable portion) may comprise a material that comprises shape memory, such as nitinol. Thus, in alternate embodiments, the delivery member(s) may be made of aluminum, stainless steel, spring steel, nickel titanium alloys, or other alloys. Or, in some embodiments, the delivery member(s) may be made of plastic. For example, a resilient plastic such as vinyl, nylon, polypropylene, a polyethylene, ionomer, polyurethane, and polyethylene tetraphthalate (PET) may be used. Also, in alternate embodiments, and depending upon the length and width of the access members, the delivery member(s) may range in diameter from about 0.1 mm to 5 mm, or from about 0.2 mm to 3 mm, or from about 1 to 2 mm.

As described herein, the expandable portion may comprise a structural member to urge the expandable portion to expand. The structural member may comprise a framework that gives rigidity to at least a portion of the expandable portion. The framework may comprise a single wire. Alternatively, the framework may comprise a plurality of wires. The framework (or members used to make up the framework) may comprise a material that is flexible, such that the framework is able to bend. Alternatively, the framework may be relatively rigid. Also, in one embodiment, the framework or portions thereof may comprise a material that comprises shape memory, such as nitinol. Thus, in alternate embodiments, the framework (or portions thereof) may also be made of aluminum, stainless steel, spring steel, nickel titanium alloys, or other alloys. Or, in some embodiments, the framework may be made of plastic. For example, a resilient plastic such as vinyl, nylon, polypropylene, a polyethylene, ionomer, polyurethane, and polyethylene tetraphthalate (PET) may be used. Also, in alternate embodiments, and depending upon the length and width of the expandable portion, the individual members used to make up the framework may comprise wires that range in diameter from about 0.1 mm to 5 mm, or from about 0.2 mm to 3 mm, or from about 1 to 2 mm.

In one embodiment, the methods and devices of the present invention may be used to section and repair a vertebral body or intervertebral disc. Thus, a percutaneous access may be used to insert two cannulas into a vertebral body (or an intervertebral disc). Next, a first elongated member comprising a wire saw with a magnet or a ferrule tip on the distal end may be passed through one of the cannulas, and a stylet with a magnet on the distal end is passed through the second cannula. As described herein, the distal end of the saw may comprise a leader wire that comprises the ferrule tip or magnet. A communication may be established between the stylet and the saw allowing the saw to be drawn through the first cannula and into the second cannula. By pulling back and forth on the saw, a portion of the vertebral body may be sectioned. At this point, the saw may be removed, and two inflatable members (e.g., balloons) may be inserted into the portion of the vertebral body that has been cut away. One balloon may be inserted via each cannula. The balloons may be expanded, such that the anterior portion of the vertebral body is lifted up until the endplates of the vertebral body are parallel. At this point, there may be a wedge-shaped opening in the vertebral body.

Using the cannulas, an expandable barrier may then be emplaced in the vertebral body. Thus, a first elongated member comprising a ferrule or magnet on the distal end and including an expandable barrier may be passed through one of the cannulas. A stylet with a magnet on the distal end may be passed through the second cannula. A communication may be established between the stylet and the distal end of the expandable barrier via the interaction of the magnet on the stylet with the ferrule on top of the expandable barrier, allowing the barrier to be drawn through the first cannula and positioned in the vertebral body. At this point, the expandable member may be manipulated to expand across the gap previously made in the anterior vertebral wall. A third cannula may then be inserted into the vertebral body (e.g., using a transpedicular access) and a bone repair material emplaced. Once the repair material has been emplaced, the access member cannulas may be removed. Also, in certain embodiments, the elongated members may be disconnected from the expandable barrier and the barrier left in place.

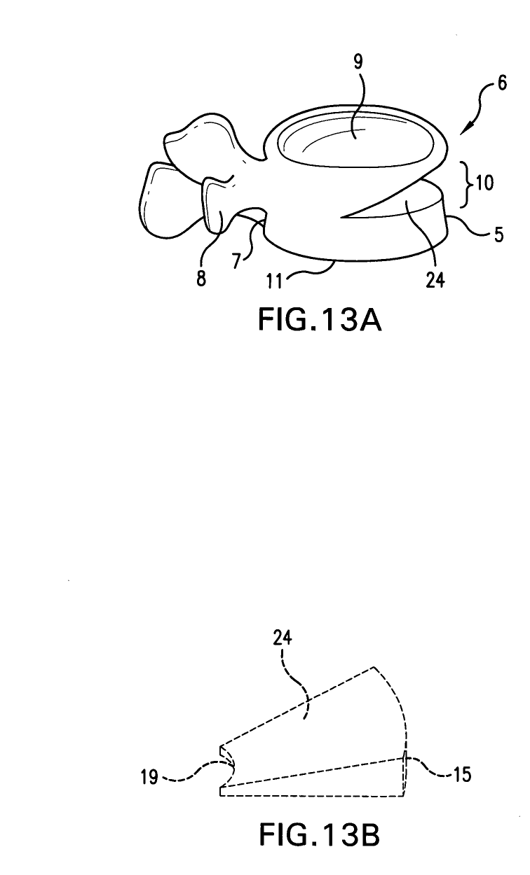

A device comprising an expandable barrier and methods of using such devices are shown in FIGS. 13-19. Although the body part shown in FIGS. 13-19 is a vertebral body, it is to be understood that the methods, devices and systems of the present invention may be used to repair other body parts.

FIG. 13 shows a side view of a vertebral body needing repair. Thus, as shown in FIG. 13, the vertebral body comprises an anterior side 5, and a posterior 7 side where the pedicles 8 and spinal cord (not shown) are located. In some cases, the vertebral body (or other bone structure) may have a gap in the body part that includes a portion of the wall 10 that needs to be replaced. For example, in one embodiment, a vertebral body may be sectioned and refractured using a sectioning saw and inflatable members (e.g., kyphoplasty balloons) as described herein. The balloons may be used to further separate the interior and superior portions of the vertebral body to achieve parallel orientation of the endplates 9, 11.

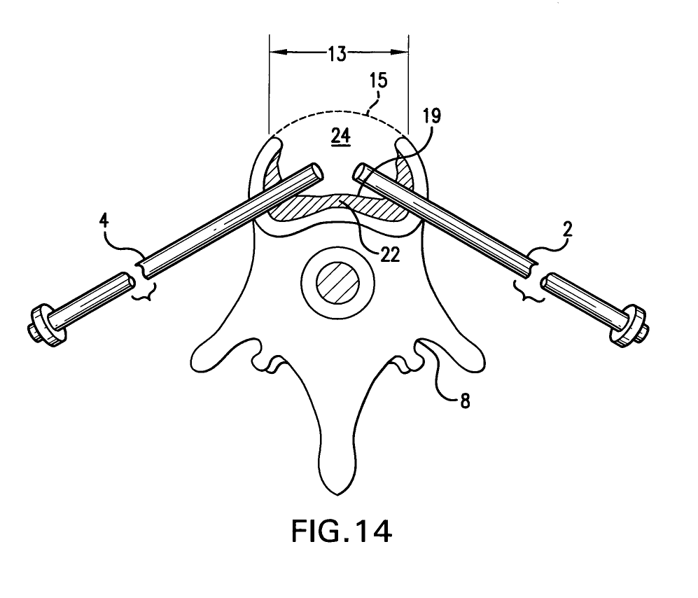

FIG. 14 shows a top view of a vertebral body that has been sectioned and expanded in a manner so as to leave a gap 13 in the anterior wall as may result upon inflation of two balloons that were introduced into a sectioned vertebral body using two access member cannulas 2, 4. Thus, upon lifting of the upper portion of the vertebral body, there may be a void 24 (also shown in FIG. 13 as the dotted area) that needs to be filled to achieve endplate parallelism. As shown in FIGS. 13 and 14, the void may be substantially triangular in cross-section such that the portion of the void nearer to the anterior wall 15 is larger than the portion of the void nearer to the posterior wall 19. If a physician was to fill the void with cement, it may be difficult to prevent the cement from leaking out of the opening 15 at the anterior wall.

In one embodiment, the present invention may comprise an expandable barrier that is emplaced in a body part having a section that needs to be closed off. For example, in one embodiment, the barrier may be used to seal the gap present in a vertebral body that has been sectioned and needs repair. Or, the barrier may be used to replace other types of breaks or areas of bone loss.

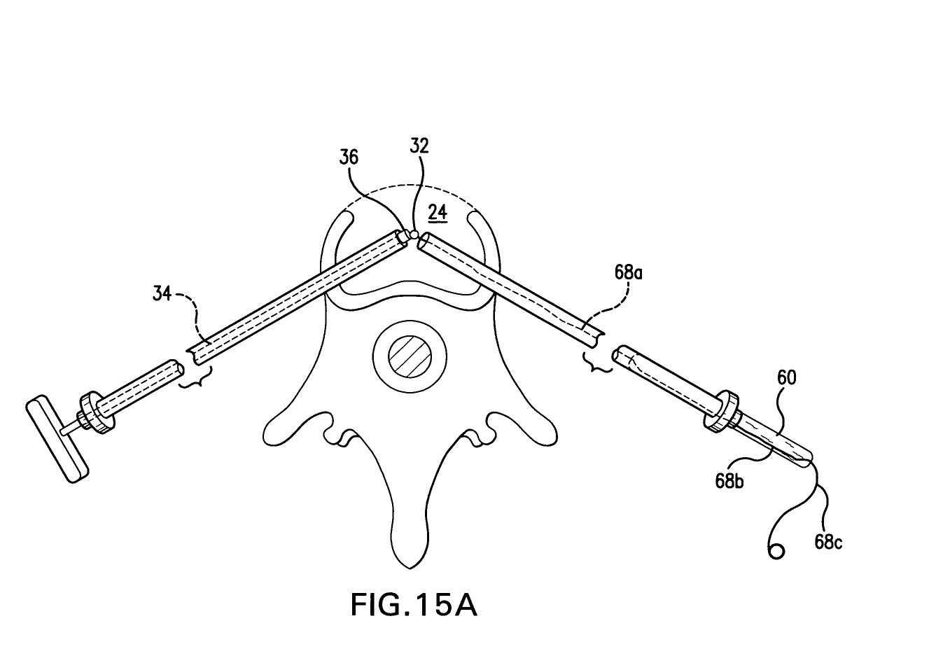

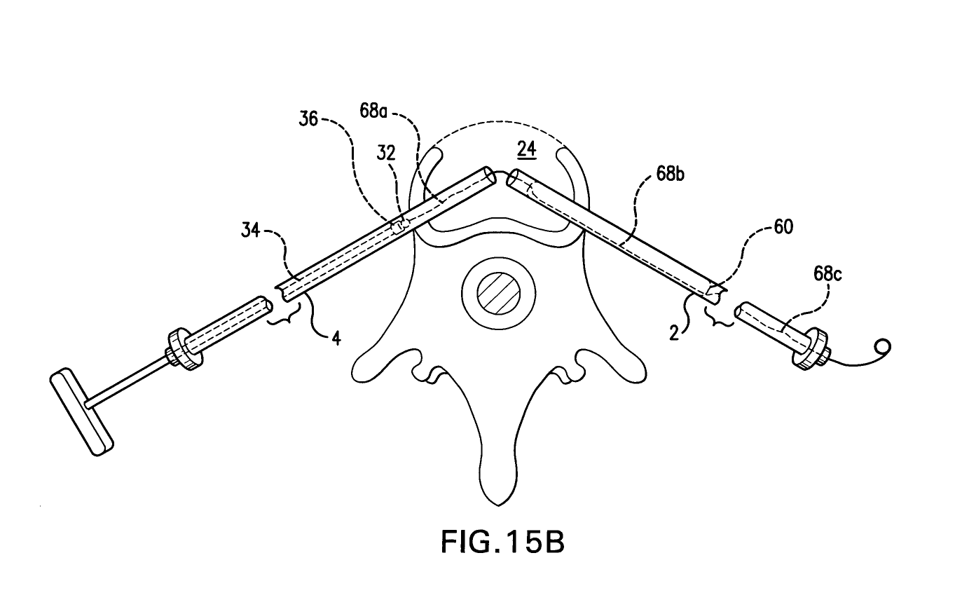

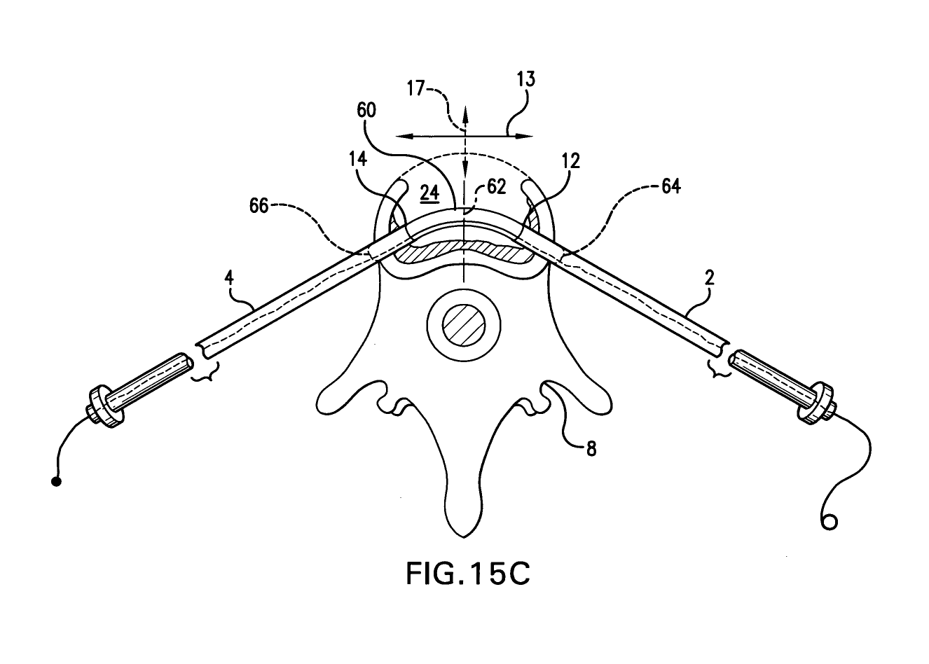

FIGS. 14 and 15 shows an embodiment of an expandable barrier 60 in a void 24 in a vertebral body using two access members 2, 4. In one embodiment, the first and second access members are positioned so as to be closely juxtaposed (FIG. 14). At this point, a stylet 34 having a rare earth magnet 36 on the distal end may be inserted in one of the access members 4 (FIG. 15A). Also, a tool comprising an expandable barrier 60, a first delivery member 68, and having a ferrule tip 32 or other type of material attracted to the magnet 36 on the distal end of the stylet 18 may be inserted into the other access member (FIG. 15A). Or, as described herein, other types of devices for engaging the distal end of the two delivery members 30, 34 may be used.

Once the distal end 32 of the expandable barrier engages the distal end 36 of the stylet 34 (FIG. 15A), the stylet may be used to pull the distal end of the expandable barrier into the second access member 4 (FIG. 15B). The expandable barrier 60 may then be pulled 40 through the first and second access members 2, 4 (FIG. 15B) until the barrier is positioned such that the midline of the barrier 62 is approximately aligned with the midline 17 of portion of the body part that needs to be patched 13 by the barrier (FIG. 15C). Thus, as shown in FIG. 15C, in one embodiment, the ends 64, 66 of the barrier 60 are each approximately equidistant from the ends 12, 14 of the first and second access members 2, 4, respectively.

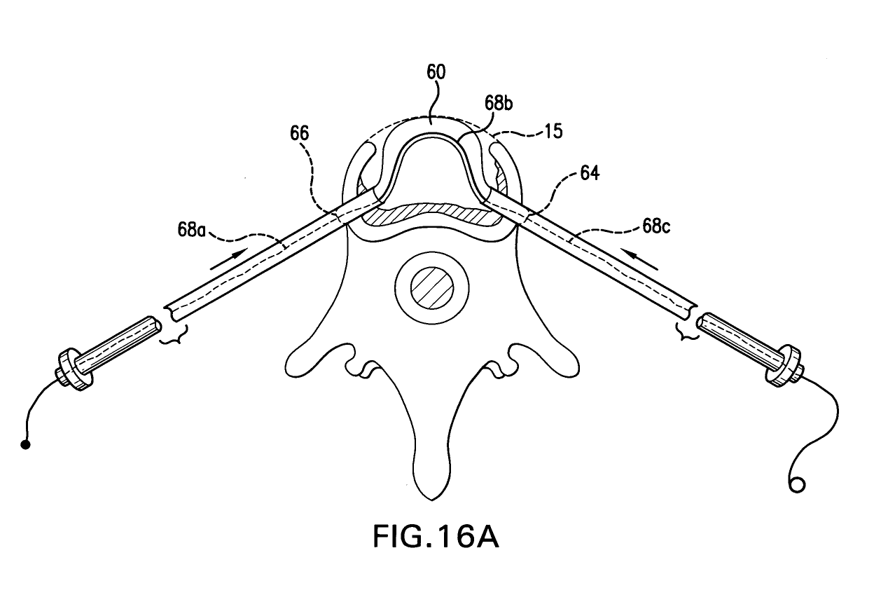

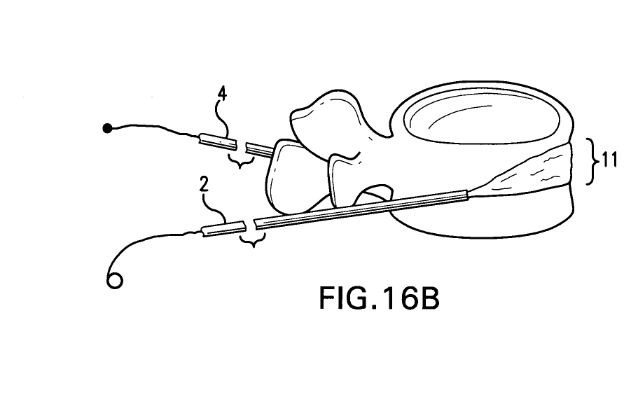

Next, the barrier may be extended into the body part so as to assume a shape that is required to cover the opening 15 in the body part (FIG. 16). For example, as shown in FIGS. 16A and 16B, the barrier 60 may be expanded so as to extend along the periphery 15 of the vertebral body, thereby replacing the missing anterior wall. Alternatively, the barrier 60 may comprise a preformed shape (such as a hemispherical shape) that is restrained while the barrier is substantially contained within the access members 2, 4 (FIG. 15A), and/or by pulling on the ends 64, 66 of the barrier 60 (FIG. 15C), but that expands upon the barrier emerging from the access members (FIG. 16).

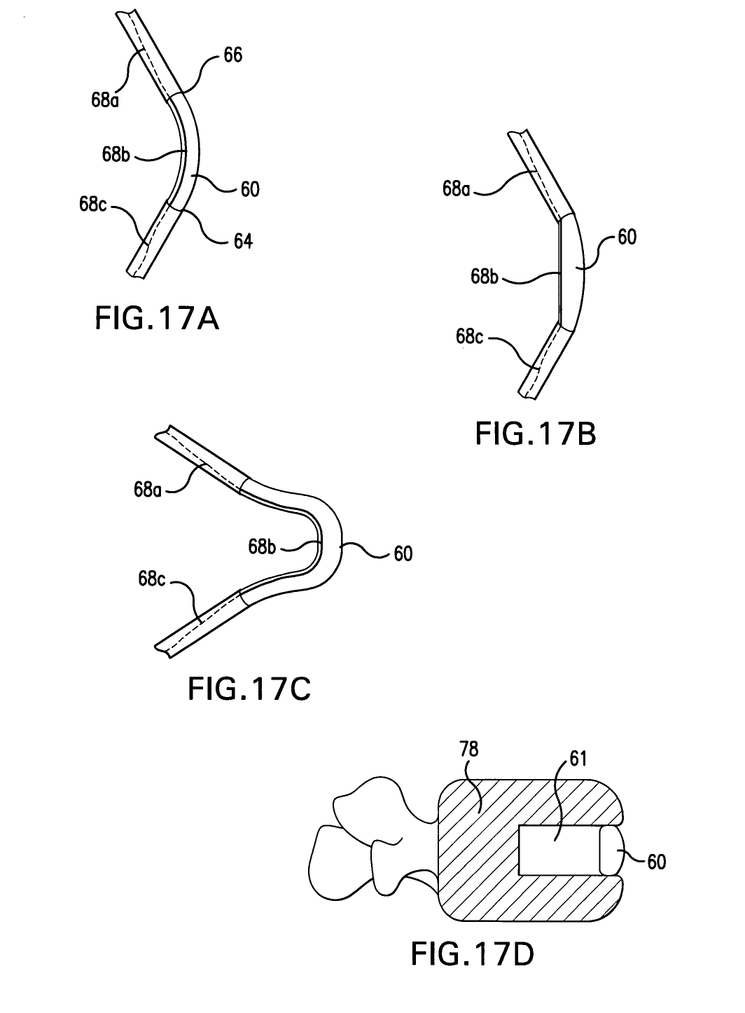

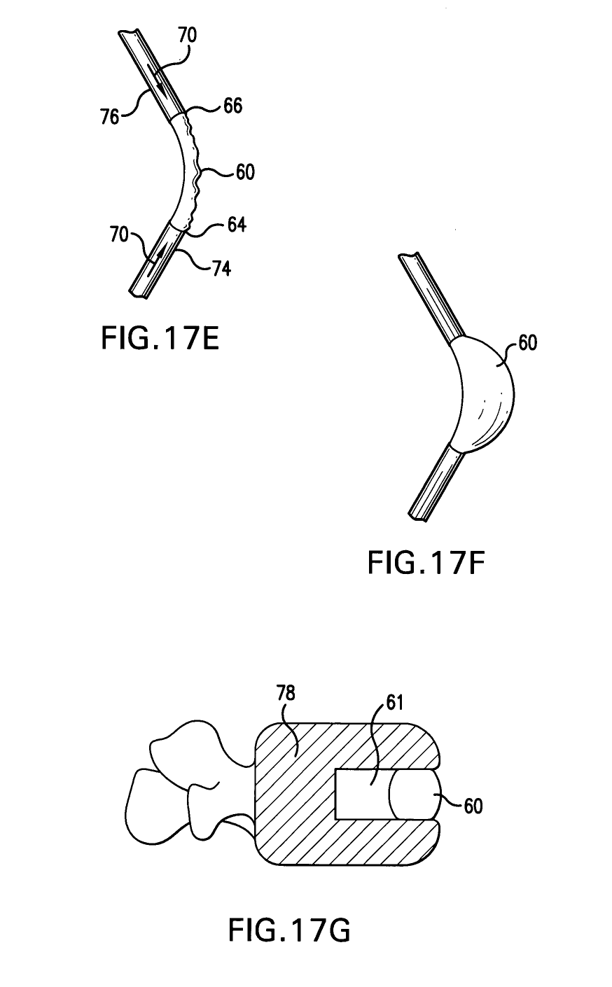

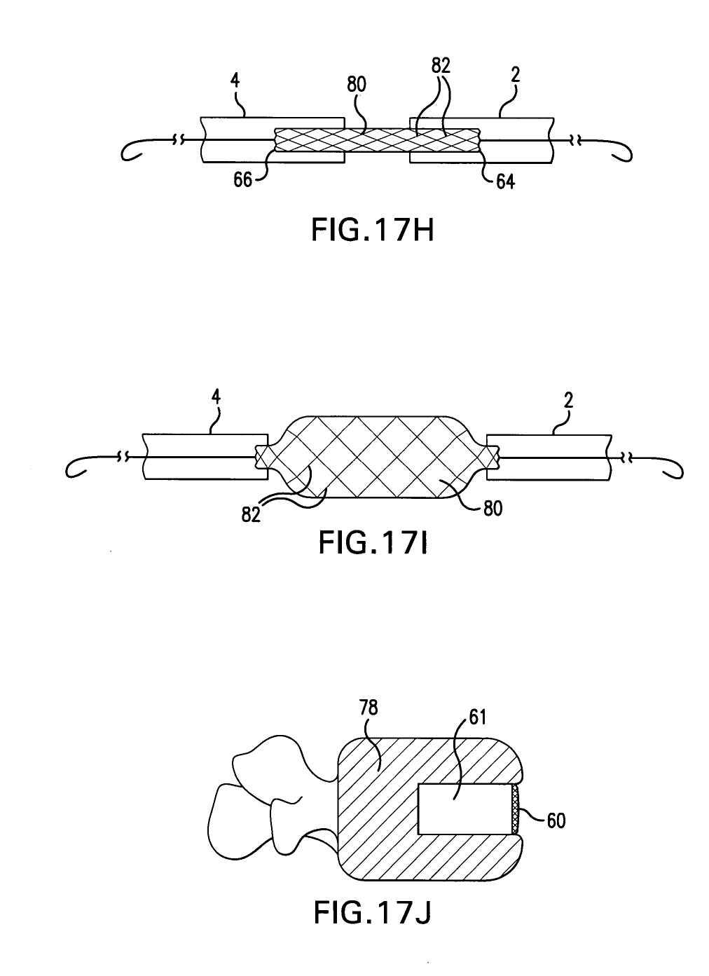

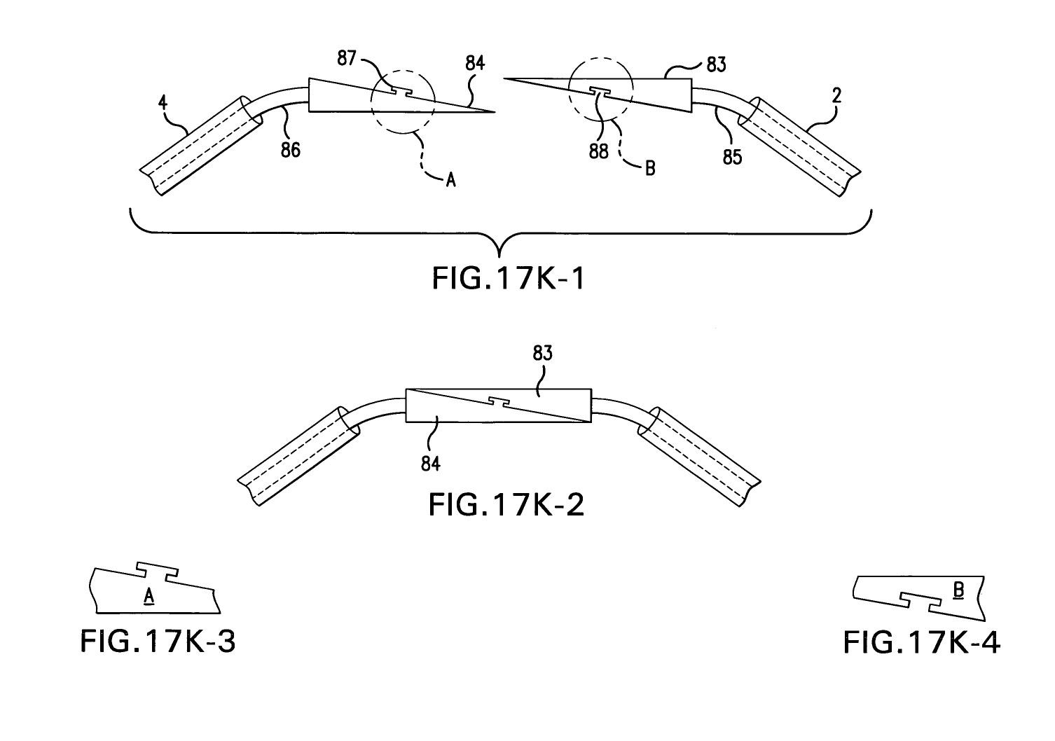

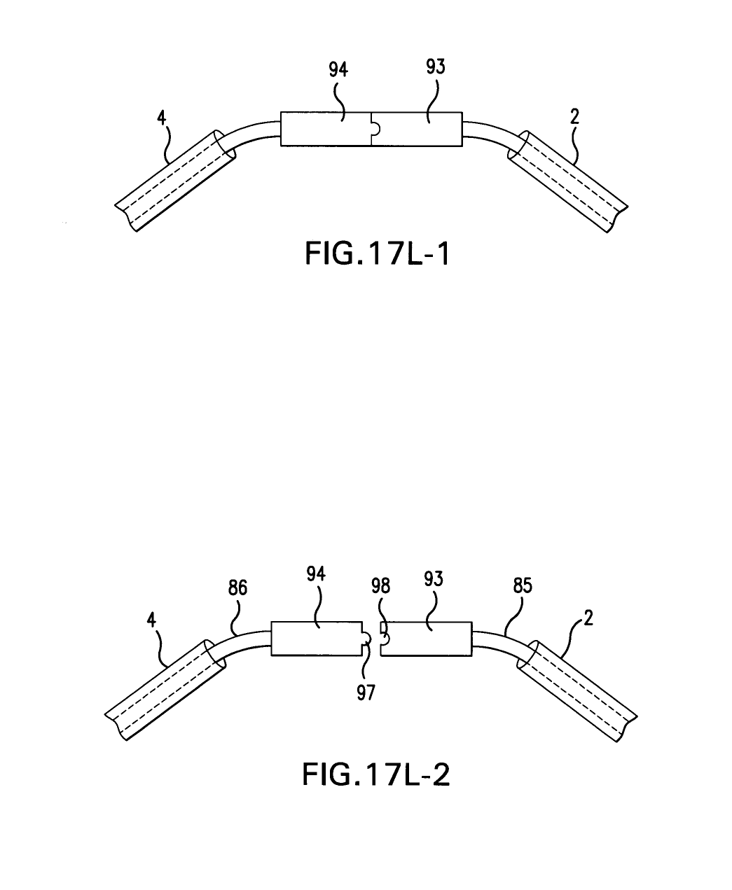

A variety of methods may be used to expand the barrier. In one embodiment, the barrier may comprise a structural member such as a wire 68 that may be used to modify the shape of the barrier. In one embodiment, the wire is fixedly attached at the ends 64, 66 of the expandable portion of the barrier 60. Thus, the wire may comprise two portions 68a and 68c that are exterior to the expandable portion 60, and one portion 68b that is inside of the expandable portion 60. In this way, by pushing the proximal ends of the wire towards the distal ends of the access members, the expandable barrier may be urged into a convex shape that extends along and covers the gap in the vertebral wall. Thus, as shown in FIG. 17A, the expandable barrier may have a preformed shape that is partially arcuate when the wires are not pulled or pushed on by the physician. If the physician pulls on the ends of the wire, the expandable barrier may assume a relatively straight configuration (FIG. 17B). If the physician pushes on the ends of the wires, the expandable barrier may comprise an even more arcuate shape (i.e., become even more expanded) (FIG. 17C). FIG. 17D shows a lateral cross-section of an expandable barrier of the present invention emplaced in a body part 78. In this embodiment, the wire may function as a delivery member and as a framework.

In another embodiment, the expandable portion of the barrier 60 may include an internal void, such that the barrier may be inflated by the introduction of a gas or a fluid. For example, the barrier may be a balloon that can be inflated with air or another gas. Alternatively, a liquid may be used to expand the barrier. Or, the barrier may be inflated with a curable rigid material (e.g., bone cement or other repair material) for permanent emplacement. FIG. 17 E shows one embodiment of an expandable membrane 60 that may be inflated by inserting a gas or liquid 70 into the membrane via access members 74, 76 to assume an expanded shape as shown in FIG. 17F. FIG. 17G shows a lateral cross-section of an inflated barrier of the present invention emplaced in a body part 78.

In yet another embodiment, the barrier may comprise a curtain-like structure that is able to unfold upon emerging from the access members. For example, the barrier may comprise a membrane 80 that includes a wire framework 82 that can unfold as the barrier emerges from the access members. In one embodiment, the first and second access members 2, 4 hold the expandable barrier in an unexpanded configuration (FIG. 17 H). As the expandable barrier is released from the access members 2, 4 the framework 82 naturally expands to urge the membrane to an expanded configuration (FIG. 17I). FIG. 17J shows a lateral cross-section of an expanded membrane barrier 80 of the present invention emplaced in a body part 78.

FIG. 17K shows an example of a two-piece barrier that may be inserted in a body part. Thus, as shown in FIG. 17K-1, one half of the barrier 84 may comprise a fixture 87 that protrudes, and interlocks with an aperture 88 on the other half of the barrier 83. The barriers may be inserted into a body part using elongated members 85 and 86, that are inserted into the body part using an access path or access members 2,4, respectively. FIG. 17K-2 shows the two pieces of the barrier interlocked, and FIG. 17K-3 and 17K-4 shows an enlargement of the two interlocking portions of the barrier.

Similarly, 17L shows an example of a two-piece barrier that may be inserted in a body part. Thus, as shown in FIG. 17L-1, one half of the barrier 94 may comprise a fixture 97 that protrudes, and interlocks with an aperture 98 on the other half of the barrier 93. The barriers may be inserted into a body part using elongated members 85 and 86, that are inserted into the body part using an access path or access members 2,4, respectively. FIG. 17L-2 shows the two pieces of the barrier interlocked.

In one embodiment, the barrier expands in three dimensions. Thus, as shown in FIGS. 16 and 17, the barrier may be expanded in the X and Y directions to extend as an arcuate shape to cover the width 13, height 10, and the peripheral boundary 15 of the body part being replaced (FIG. 16A). The barrier may also be expanded in the Z direction (e.g., parallel to the spinal cord for a disc or vertebral body) so as to cover the height 11 of the opening as shown in FIG. 16B.

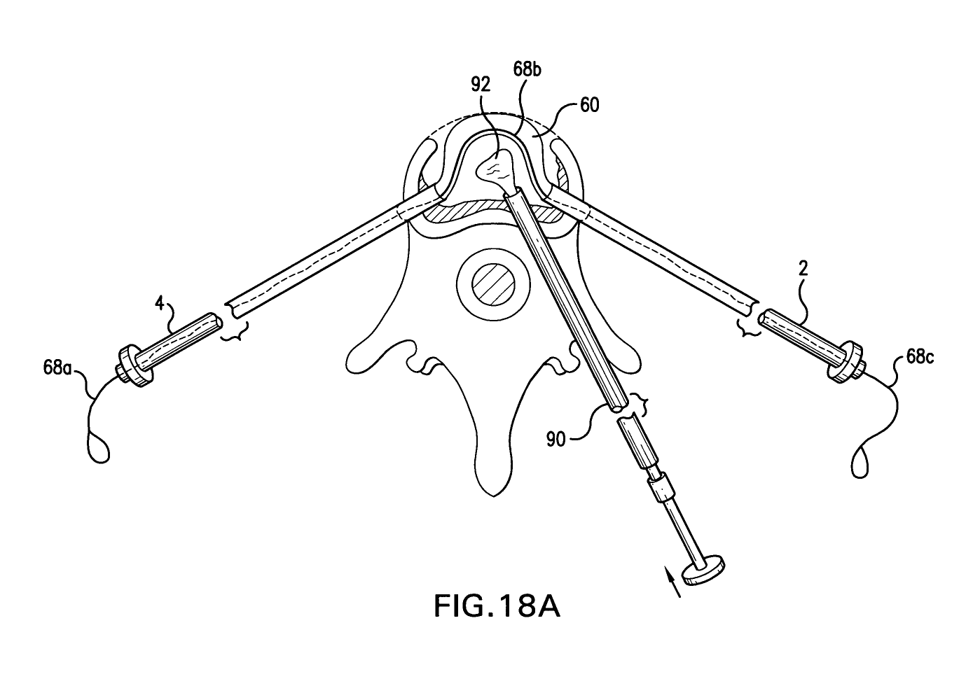

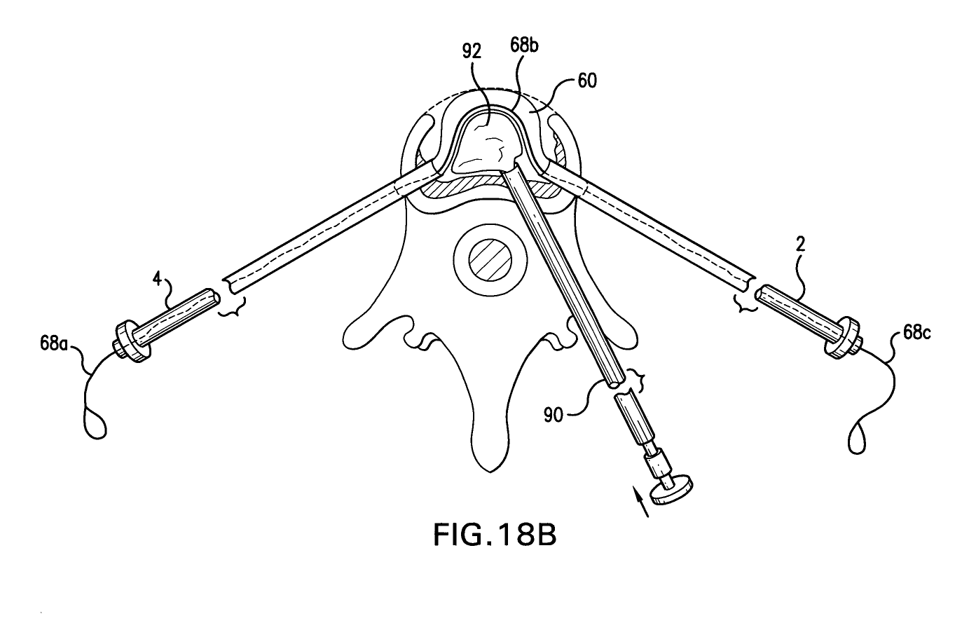

Once the barrier is in place, the body part may now comprise a closed off area (e.g., a cavity) that can be filled with cement or other repair material. At this point, the cavity may be filled with the material required. For example, where the expandable barrier is used to repair a vertebral body, a third access member 90 may be inserted into the vertebral body and a cement or bone repair material 92 delivered via the third access member (FIG. 18A). The material may be delivered so as to fill the void in the body part and extend against the expandable barrier 60 (FIG. 18B).

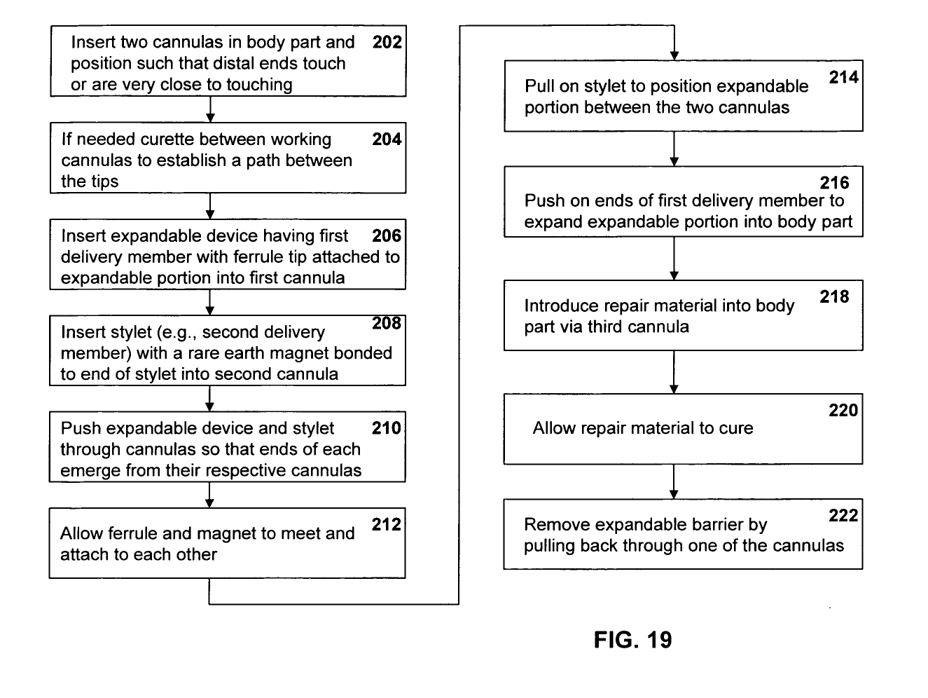

FIG. 19 shows one embodiment of a method of emplacing an expandable barrier of the present invention. FIG. 19 provides an overview of a method of using a multipoint access device to access a body part. Although the example shown as FIG. 19 is the use of an expandable device to repair a vertebral body, the methods and devices may be used for other procedures known in the art of bone repair. For example, an expandable device of the present invention may be used to repair an intervertebral disc or other bones or body parts.

Thus, as shown in FIG. 19, in one embodiment, the expandable device may be emplaced using two cannulas positioned for an extrapedicular approach to access a vertebral body. The cannulas may be inserted in such a manner so as to straddle the medial plane close to the anterior cortical wall. In one embodiment, the working cannulas are superimposed in the lateral view, or at least have their tips laterally superimposed 202. At this point, the operator may insert a stylet having a curette at one end into one of the cannulas and push the stylet through the cannula such that the blade emerges from the distal end of the cannula. The curette blade may be used to clear a path between the distal ends of the two cannulas 204 if there is not an opening already present in the body part to be repaired.

Next, the cannulas may be positioned to be even closer to each other at the distal ends, and an expandable device may be inserted into the end of one of the cannulas and threaded through the cannula such that the end of the expandable device begins to emerge from the distal end of the cannula. As described above, the expandable device may comprise a first delivery member that has a magnet (e.g., a rare earth magnet) or a ferrule tip 206. Also, a second delivery member (e.g. stylet) having a magnet at the distal end may be inserted into the second cannula and threaded through the cannula such that the end of the stylet begins to emerge from the distal end of the cannula (208). When both the stylet and the expandable device emerge from the distal ends of their respective cannulas (210), the magnet will draw the ferrule towards it, thereby linking the ferrule to the stylet (212). In this way, a communication may be established between the two cannulas. At this point, the distal portion of the expandable device may be drawn up through the second cannula to a point that allows the expandable portion to be positioned in the body part by pulling the stylet out of the proximal end of the second cannula (214).

Next, the operator may expand the expandable portion in the body part. For example, in one embodiment, the operator may push on the distal ends of the first delivery member to expand the expandable barrier so that the barrier extends along the periphery of the anterior wall of the vertebral body 218. At this point, a repair material may be introduced into the vertebral body 218 and allowed to cure 220. Once the repair material has cured, the expandable barrier may be removed from the body part by pulling the barrier back through one of the cannulas (222).

### Systems and Kits

In other embodiments, the present invention may comprise a system. For example, in certain embodiments, the system may comprise a surgical or medical kit. The kit may be suitable for medical or veterinary use, as for example, for emplacement of a material in a human, or an animal.

In certain embodiments of the systems and kits of the present invention, the parts may be configured for accessing a body part within a living body. In one embodiment, the body part may comprise a bone. In one embodiment, the predetermined location may comprise a bone interior. For example, the predetermined location may comprise a portion of a spine. Thus, in one embodiment, the components of the systems and kits of the present invention may be fashioned for accessing a vertebral body or disc of a spine.

As described herein, the access path may comprise a percutaneous access to the spine. For example, the first and second access members may be configured for accessing the spine. In certain embodiments, the first and second access members are configured for accessing the vertebral body. Or, the first and second access members are configured for accessing the vertebral body. The first and second members may be configured to access the spine by percutaneous access. The body part may comprise a vertebral body and/or an intervertebral disc. Thus, in an embodiment, an access member cannula may be used to access a vertebral body or a spinal disc. Where the body part is a vertebral body, the two entry points may be either transpedicular or extrapedicular. Thus, in one embodiment, the first access member may be configured for accessing interior of a vertebral body through a first pedicle of the vertebral body, and the second access member may be configured for accessing interior of the vertebral body through a second pedicle of the vertebral body. Once the access members are inserted into the body part, the distal ends of the first and second access members may be positioned adjacent to a portion of the body part requiring sectioning.

For example, as described herein, due to various traumatic or pathologic conditions, such as osteoporosis, a vertebral body can experience a vertebral compression fracture (VCF). Thus, the systems and kits of the present invention may by used to repair a vertebral body or an intervertebral disc. In an embodiment, the systems and kits are configures to repair a spinal tissue that is lost due to a fracture or other degeneration, or to remove excess sclerotic bone from a vertebral body. The systems and kits of the present invention may also be used to repair other parts of a living or non-living organism. For example, in certain embodiments, the kits and systems of the present invention can be deployed in other bone types and within or adjacent other tissue types, such as an arm bone, a leg bone, a knee joint, or the like.

For example, in one embodiment, the present invention may comprise a surgical kit comprising a device for multipoint access of a body part comprising: (a) a first access member; (b) a second access member; (c) a first inner member to be inserted into the first access member; and (d) a second inner member to be inserted into the second access member, wherein the distal end of the first inner member is able to engage the distal end of the second inner member. In one embodiment, the access member may comprise a path for delivering the material to the body part of interest. The access members may be configured to provide percutaneous access to a portion of a spine, such as a vertebral body or an intervertebral disc.

There may be a variety of methods whereby the first and second inner members may be connected or engaged. In one embodiment, the end of one inner member may comprise a magnet, and the end of the other inner member may comprise a material that binds to a magnet. For example, in one embodiment, the distal surface of the second inner member may comprise a magnet (e.g., a rare earth magnet), and the distal surface of the first inner member may comprise a material that binds to the rare earth magnet. In one embodiment, the distal surface of the second inner member comprises a ferrous material or a magnet. Or, in one embodiment, the distal surface of the first inner member may comprise a magnet and the distal surface of the second inner member may comprise a material that binds the magnet.

In other embodiments, a fastening device may be used to engage the two inner members and establish a communication between the first and second access members. For example, the distal end of one inner member may comprise a hook and the distal end of the other inner member may comprise a loop. Or, the distal end of one inner member may comprise a protrusion (e.g., a male fitting) that fits within an aperture or well (e.g., a female fitting) on the end of the other inner member. Or, in one embodiment, one of the inner members may comprise a micro-rongeur or a collapsible cage that is able to couple the distal end of the other inner member. Or, chemical coupling may be used as described herein.

In some embodiments, the first inner member may comprise a tool to repair the body part. For example, the first inner member may comprise a cutting tool. In one embodiment, the first member may comprise a wire saw known as a giggly saw. Or, other types of cutting devices known in the art may be used. In one embodiment, the wire saw may be used to cut through or section a portion of the body part. In another embodiment, the first inner member comprises an expandable barrier as described in further detail below.

In one embodiment, an inner member may comprise a material that is substantially rigid. For example, in certain embodiments, at least one inner member may comprise a rigid rod or a stylet. Or, the inner member may be bendable such as a wire saw. Or, the inner member may also comprise portions that are substantially rigid combined with portions that are bendable. In certain embodiments, the inner member(s) may comprise a material that comprises structural or shape memory, such as nitinol. Thus, in alternate embodiments, the inner member(s) may be made of aluminum, stainless steel, spring steel, nickel titanium alloys, or other alloys. Or, in some embodiments, the inner member(s) may be made of plastic. For example, a resilient plastic such as vinyl, nylon, polypropylene, a polyethylene, ionomer, polyurethane, and polyethylene tetraphthalate (PET) may be used. Also, in alternate embodiments, and depending upon the length and width of the access members, the inner member(s) may range in diameter from about 0.1 mm to 5 mm, or from about 0.2 mm to 3 mm, or from about 1 to 2 mm.

The access member used with the systems and kits of the present invention may provide a path to access a region or a body part that is located within a subject's body. The access member may be any type of device that can extend from the location of interest (e.g., a bone or an organ) to be accessible to a user of the access member. For example, the access member may be designed to extend from an internal body part in a subject to outside of the subject's body. The access member may comprise an elongated hollow member such as a hollow cylinder or a tube. Thus, in one embodiment, the tube may be designed to provide an access from outside of the living body to the internal body part. For example, the access member may comprise a cannula, such as a cannula used to deliver a material to bone or another type of body part.

In certain embodiments, the access member of the systems and kits of the present invention may be configured to provide percutaneous surgical access from outside of the subject to the predetermined location. In an embodiment, the body part being accessed is the spine. In alternate embodiments, the percutaneous surgical access may comprises an incision ranging from about 0.1 to 4.0 centimeters (cm) in diameter, or from about 0.2 to 2.0 cm in diameter, or from about 0.25 to about 1 cm in diameter. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter. For example, in a typical percutaneous surgical repair of a spine, a cannula may establish a percutaneous path along its elongated axis to a vertebral body of one of the several vertebrae.

In one embodiment, the access members are cylindrical in shape. Or, the access member may be other shapes, such as oval, rectangular, polygonal (e.g., hexagonal, octagonal) and the like. For example, in one embodiment, the access member comprises a cannula. Also, the access members may comprise a material that is compatible with the other parts of the system. For example, metals such as stainless steel, spring steel, nickel titanium alloys, other alloys, or aluminum may be used. Or, the access members may be made of plastic. For example, a resilient plastic such as vinyl, nylon, polypropylene, polyethylene, ionomer, polyurethane, or polyethylene tetraphthalate (PET) may be used. Again, one of ordinary skill in the art having the benefit of this disclosure would appreciate that other materials, including those that are well-known to one in the art, may be applied to configure the access members described herein.

In one embodiment, the kit may comprise an inner seal, comprising an inner wrap, that may be sealed by heat or vacuum, to prevent the components of the kit from being exposed to the outside environment. The inner seal may comprise a conventional peal-away seal to provide quick access to the components of the kit. Also, in one embodiment, the kit may include an outer wrap, also sealed by heat or the like, to enclose the inner wrap. Like the inner seal, the outer seal may comprise a conventional peal-away seal to provide quick access to the components of the kit. Use of an outer wrap may allow the kit to be prepared for imminent use by removing the outer wrap while leaving the inner wrap in place to ensure sterility of the kit components. The kit may also comprise a case to protect the components of the system from physical damage. In one embodiment, the outer wrap may be made of materials commonly used in the art such as polyethylene and MYLAR™, to allow for visualization of the components in the kit. The inner wrap may be made of materials such as TYVEK™ (DUPONT®), that is permeable to ethylene oxide (ETO) sterilizing gas. Sterilization may be by heat, pressure and/or sterilization gas as is known in the art. Also, the kit may include directions for use by a physician or other trained personnel.

Embodiments of the system and/or kits of the present invention may further comprise a material to be delivered to the internal body part. In one embodiment, the material to be delivered to the body part may be emplaced within at least a portion of at least one access member. For example, where the system is being used for bone repair, a tube may be loaded with a bone filler material or an adhesive. In one embodiment, the bone filler material may comprise a mixture containing calcium, hydroxyl apatite, and a polymer. Also, in certain embodiments, the bone filler may comprise ceramic granules or other filler material.

In other embodiments, the material may comprise an autograft or allograft bone graft tissue (see e.g., Dick, Archives of Orthopaedic and Traumatic Surgery (1986), 105: 235-238; or Bhan et al, International Orthopaedics (SICOT) (1993) 17: 310-312). The bone graft tissue can be obtained using a Bone Graft Harvester, which is commercially available from SpineTech. Alternatively, the material may also comprise a granular bone material harvested from coral, e.g., PROOSTEON™ calcium carbonate granules, available from Interpore. The granules may be loaded into the access member using a funnel or other loading means. The material for delivery to a bone can also comprise demineralized bone matrix suspended in glycerol (e.g., GRAFTON™ allograft material available from Osteotech), or SRS™ calcium phosphate cement available from Novian. The material for delivery to a bone can also be in sheet form, e.g., COLLAGRAFT™ material made from calcium carbonate powder and collagen from bovine bone. In one embodiment, the sheet may be rolled into a tube and loaded by hand into the access member. In one embodiment, KYPHX®HV-R™ bone cement, commercially available from Kyphon, Inc., may be used.

In some embodiments, the present invention comprises kits or systems comprising a device to be emplaced in a body part. In an embodiment, the body part is spinal tissue, such as an intervertebral disc or a vertebral body. In an embodiment, the device comprises an expandable member. In an embodiment, at least a portion of the device may be configured so as to be able to alter the device from a first configuration into a second configuration. Where the device is expandable, the first configuration may be an unexpanded configuration and the second configuration may be an expanded configuration. Alternatively, the device may comprise a cutting member. The components may be configured for percutaneous access as described herein. Also, the device may be configured to access a vertebral body or disc. In an embodiment, the device may be configured so as to be able to be disconnected from any delivery members used to emplace the device. For example, the device may be connected to the delivery members using connectors that may be separated.

Yet other embodiments of the systems or kits of the present invention may comprise an expandable device for emplacement in a body part. In certain embodiments, the expandable device may comprise an expandable portion comprising a surface having a first unexpanded shape and a second expanded shape. Also, the expandable device may comprise a delivery member for delivering the expandable portion to a body part. As described above, the expandable device may be designed to be accessed by an operator at two different places on the device. For example, in some embodiments, the expandable device may comprise an expandable portion having a first end and a second end, and a first delivery member for delivering the expandable portion to a body part, wherein the first delivery member comprises a first part attached to the one end of the expandable portion and a second part attached to the second end of the expandable portion. In other embodiments, a multipiece barrier as described herein may be included in the kits or systems of the present invention.

The expandable device may further comprise a means to deliver the expandable device to a body part. Thus, in one embodiment, the expandable device may comprise a delivery system. In one embodiment, the delivery system comprises a first access member and a second access member. The delivery system may also comprise a second delivery member to be inserted into the second access member. In one embodiment, the distal end of the delivery member attached to the expandable portion comprises a first binding component that can engage and bind to a second binding component on the distal end of the second delivery member. Thus, in certain embodiments of the systems and kits of the present invention, the delivery system may comprise a means to have the first and second delivery members interact, to thereby form a communication between the first and second delivery members such that the expandable member can be positioned in the body part of interest.

A variety of methods may be used to establish a communication between the two delivery members used in the systems and kits of the present invention. For example, in one embodiment, the distal end of the second delivery member comprises a magnet (e.g., a rare earth magnet), and the distal end of the first delivery member attached to the expandable portion comprises a material that binds to the magnet. For example, the distal end of the first delivery member may comprise a ferrous material or a magnet. Or, in one embodiment, the distal end of the first delivery member may comprise a magnet and the distal end of the second delivery member may comprise a material that binds the magnet.

In other embodiments, a fastening device may be used to engage the two delivery members. For example, the distal end of one delivery member may comprise a hook and the distal end of the other delivery member may comprise a loop. Or, the distal end of one delivery member may comprise a protrusion (e.g., a male fitting) that fits within an aperture or well (e.g., a female fitting) on the end of the other delivery member. Or, in one embodiment, one of the delivery members may comprise a micro-rongeur that is able to couple the distal end of the other delivery member. Or a collapsing cage may be used. Or, as described herein, the ends of the delivery members may be coated with a chemical agent that when activated allows for the two delivery members to bind to each other.

There may be a variety of methods whereby the expandable device may be expanded. In one embodiment, the expandable portion may comprise a structural member that can urge the expandable portion from a first unexpanded shape to a second expanded shape. In one embodiment the structural member is attached to at least a portion of the expandable portion. In an embodiment, the structural member comprises a framework. In one embodiment, the framework may comprise a single wire. In other embodiments, the framework may comprise a plurality of wires.

The expandable portion used for the expandable device in the systems and kits of the present invention may be made of any material that can be functionally expanded in the body part of interest. For example, the expandable portion may comprise a membrane. Alternatively or additionally, the expandable device may comprise an inflatable membrane. Thus, in certain embodiments, the expandable device may be expanded by the introduction of a gas or fluid into the expandable membrane. In one embodiment, the expandable device may comprise a biodegradable membrane. In yet another embodiment, a multi-piece barrier as described previously may be used.

The delivery member(s) used to emplace the expandable member may comprise a material that is flexible, such that the delivery member(s) are able to bend. Alternatively, the delivery members may be relatively rigid. Also, in one embodiment, the delivery member(s) (e.g., the first delivery member attached to the expandable portion) may comprise a material that comprises shape memory, such as nitinol. Thus, in alternate embodiments, the delivery member(s) may be made of aluminum, stainless steel, spring steel, nickel titanium alloys, or other alloys. Or, in some embodiments, the delivery member(s) may be made of plastic. For example, a resilient plastic such as vinyl, nylon, polypropylene, a polyethylene, ionomer, polyurethane, and polyethylene tetraphthalate (PET) may be used. Also, in alternate embodiments, and depending upon the length and width of the access members, the delivery member(s) may range in diameter from about 0.1 mm to 5 mm, or from about 0.2 mm to 3 mm, or from about 1 to 2 mm.

As described herein, the framework may comprise a single wire. Alternatively, the framework may comprise a plurality of wires. The framework (or members used to make up the framework) may comprise a material that is flexible, such that the framework is able to bend. Alternatively, the framework may be relatively rigid. Also, in one embodiment, the framework or portions thereof may comprise a material that comprises shape memory, such as nitinol. Thus, in alternate embodiments, the framework (or portions thereof) may also be made of aluminum, stainless steel, spring steel, nickel titanium alloys, or other alloys. Or, in some embodiments, the framework may be made of plastic. For example, a resilient plastic such as vinyl, nylon, polypropylene, a polyethylene, ionomer, polyurethane, and polyethylene tetraphthalate (PET) may be used. Also, in alternate embodiments, and depending upon the length and width of the expandable portion, the individual members used to make up the framework may comprise wires that range in diameter from about 0.1 mm to 5 mm, or from about 0.2 mm to 3 mm, or from about 1 to 2 mm.

In other embodiments, the present invention may comprise a method of providing a device, system or kit for bilateral access and repair of a body part in a subject. For example, in one embodiment, the method may comprise providing a device for multipoint access of a body part. In one embodiment, the method may comprise providing a device comprising a first access member and a second access member. The method may further comprise providing a first inner member to be inserted into the first access member. Also, the method may comprise providing a second inner member to be inserted into the second access member, wherein the distal end of the first inner member is configured so as to be able to engage the distal end of the second inner member.

Thus, in certain embodiments, a method of providing a device or system of the present invention may comprise the step of attaching a first binding component to one end of the first inner member. Additionally, the method may comprise attaching a second binding component to one end of the second inner member. In one embodiment, the first binding component is fashioned so as to be able to bind to the second binding component.

In one embodiment, a magnet may be attached to the end of one inner member. Also, a material that can bind to a magnet may be attached to the end of the other inner member. For example, in one embodiment, the distal end of the second inner member may be configured to comprise a rare earth magnet, and the distal end of the first inner member may configured comprise a material that binds to the rare earth magnet. In one embodiment, the distal end of the first inner member may be configured to comprise a ferrous material. Or, in one embodiment, the distal end of the first inner member may comprise a magnet and the distal end of the second inner member may comprise a material that binds the magnet. Materials that may be used as magnets and for binding to magnets are as described previously.

In other embodiments, a fastening device may be used to engage the two inner members and establish a communication between the first and second access members. For example, the distal end of one inner member may be configured to comprise a hook and the distal end of the other inner member may be configured to comprise a loop. Or, the distal end of one inner member may be configured to comprise a protrusion that fits within an aperture or well that is configured on the end of the other inner member. Or, in one embodiment, one of the inner members may comprise a micro-rongeur that is able to couple the distal end of the other inner member.

Another embodiment may comprise forming the second inner member as a collapsible claw or basket. This claw can be made from aluminum, stainless steel, spring steel, nickel titanium alloys, or other alloys. Or, in some embodiments, the claw may be made of plastic. For example, a resilient plastic such as vinyl, nylon, polypropylene, a polyethylene, ionomer, polyurethane, and polyethylene tetraphthalate (PET) may be used. The first inner member may be configured as a flexible, string-like member and have a ferrule, possibly spherical in shape, bonded to its distal end. In an embodiment, the device may also be configured to comprise a sheath that may be translated over the second inner member thereby collapsing the claw around the ferrule. Or, an actuating device, such as a wire may be used to collapse the cage. Or, chemical coupling may be used as described herein.

In yet another embodiment, the method may comprise providing an expandable device for emplacement in a body part. In certain embodiments, the expandable device may comprise an expandable portion comprising a surface having a first unexpanded shape and a second expanded shape. Also, the expandable device may comprise a delivery member for delivering the expandable portion to a body part. In certain embodiments, the expandable device may be configured to be accessed by an operator at two different places on the device. For example, in some embodiments, the expandable device may be configured to comprise an expandable portion having a first end and a second end, and a first delivery member for delivering the expandable portion to a body part, wherein the first delivery member comprises a first part attached to the one end of the expandable portion and a second part attached to the second end of the expandable portion.

The expandable device may further comprise a delivery system to deliver the expandable portion to a body part. In one embodiment, the delivery system comprises a first access member and a second access member. The delivery system may also comprise a second delivery member to be inserted into the second access member. In one embodiment, the distal end of the first delivery member attached to the expandable portion is configured to include a first binding component that can engage and bind to a second binding component that is configured on the distal end of the second delivery member. For example, in one embodiment, the distal end of the second delivery member comprises a rare earth magnet, and the distal end of the first delivery member attached to the expandable portion comprises a material (e.g., a ferrule tip) that binds to the rare earth magnet. Or, in one embodiment, the distal surface of the first delivery member may be configured comprise a magnet and the distal surface of the second delivery member may be configured comprise a material that binds the magnet.

In other embodiments, a fastening device may be used to engage the two delivery members. For example, the distal end of one delivery member may be configured to comprise a hook and the distal end of the other delivery member may comprise a loop. Or, the distal end of one delivery member may be configured to comprise a protrusion (e.g., a male fitting) that fits within an aperture or well (e.g., a female fitting) on the end of the other delivery member. Or, in one embodiment, one of the delivery members may be configured to comprise a micro-rongeur that is able to couple the distal end of the other delivery member. Or, an actuating device, such as a wire may be used to collapse the cage. Or, chemical coupling may be used as described herein.

There may be a variety of methods whereby the expandable device may be expanded. In one embodiment, the expandable portion may be configured to comprise a structural member that urges the expandable portion from a first unexpanded shape to a second expanded shape. To expand an expandable membrane, the structural member may be attached to at least a portion of the expandable portion. In one embodiment, the structural member is a framework. In one embodiment, the framework may comprise a single wire. In other embodiments, the framework may comprise a plurality of wires.

Alternatively, or additionally, the expandable device may comprise an inflatable membrane. Thus, in certain embodiments, the expandable device may be expanded by the introduction of a gas or fluid into the expandable membrane. For example, membranes such as PET and Nylon may be used. Also as discussed herein, in one embodiment, the expandable device may comprise a biodegradable membrane. Biodegradable membranes that may be used may comprise Poly(lactic acid) (PLA), Poly(glycolic acid) (PGA), Polydioxanone (PDS) or a copolymer.

The dimensions of the components made for use with the methods, devices and systems or kits described herein may vary depending upon the body parts requiring access and repair. Thus, where the devices, methods, systems or kits are used for bone repair, an access member (e.g., a cannula for accessing the bone) may comprise dimensions on the order of about 2 to 20 inches (50.8 mm to 508 mm), or about 4 to 15 inches (101 mm to 381 mm), or about 6 to 12 inches (152 mm to 305 mm), or about 8 inches (203 mm) in length. Also, in alternate embodiments, the cannula may range from about 0.05 to 0.5 inches (1.27 mm to 12.7 mm), or from about 0.1 to 0.3 inches (2.5 mm to 7.6 mm), or from about 0.12 to about 0.2 inches (3.1 mm to 5.1 mm), or from about 0.140 to 0.160 inches (3.6 mm to 4.1 mm) in diameter. The inner member (e.g. stylet) may comprise a total length that is about the same as the cannula, with a slightly smaller diameter, e.g., about 0.04 to 0.45 inches (1.0 mm to 11.4 mm), or from about 0.09 to about 0.29 inches (2.3 mm to 7.4 mm), or from about 0.1 to about 0.19 inches (2.5 mm to 4.8 mm), or from about 0.13 to 0.15 inches (3.3 mm to 3.8 mm). For example, the stylet may be sized to fit pre-existing access cannulas such as the KYPHX® Osteo Introducer device that is commercially available from Kyphon, Inc.

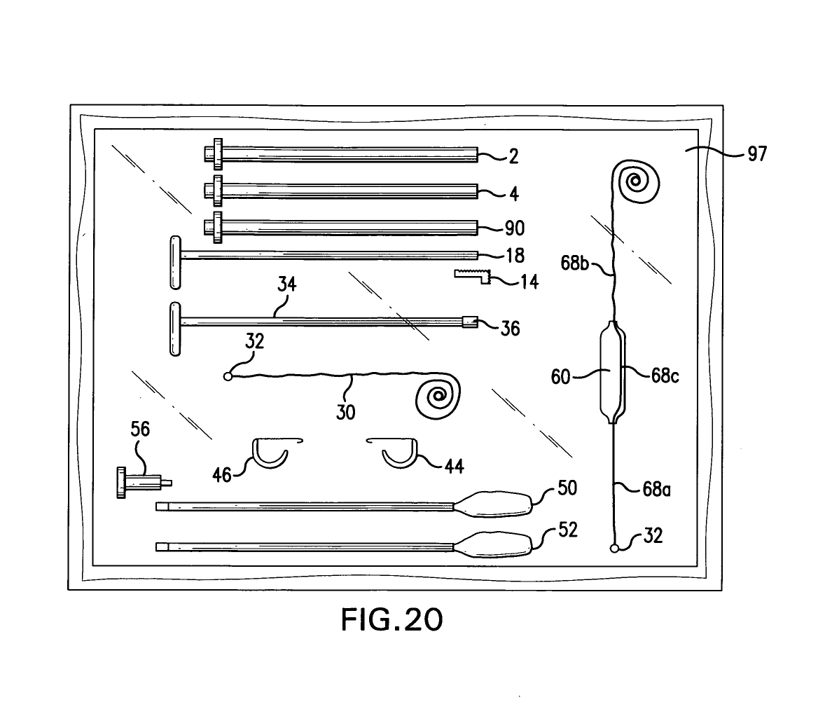

FIG. 20 shows an example embodiment of a kit of the present invention. In one embodiment, the kit may comprise a plurality of access members 2, 4 for accessing a body part. Also, the kit may comprise an access member 90 for the introduction of a repair material as described herein.

Also, the kit may comprise a first inner member. In one embodiment, the first inner member may comprise a tool for repair of the body part. For example, the first inner member may comprise a wire saw 30 as described herein.

The kit may also comprise a second inner member 34. In one embodiment, the second access member may comprise a stylet. Also, the kit may comprise a curette blade 16 that can be attached to the distal end of the second inner member. Alternatively, the kit may comprise more than one additional inner member for use with the curette. For example, in one embodiment, the curette may be attached to a second stylet 18 for use in making a path so as to establish a communication between two access members 2, 4 inserted in a body part.

In some embodiments, the kit may comprise handles 44, 46 that may be attached to the ends of either the first or second inner members or the first and second delivery members. In one embodiment, at least one of the handles is a separate part, so the handle may be attached to the first member after the first member has been pulled through the two access members. Or, the handles may comprise a collapsible loop that can function as the distal ends of the first member. Such a loop may have a ferrule fixture attached to the end of the loop. The handles may be made of any sturdy, biocompatible material. For example, the handles may be made of metal such as aluminum, stainless steel, spring steel, nickel titanium, or other metal alloys. Or, the handle may be made of a hard plastic, such as polyurethane, polystyrene polycarbonate, or nylon.

Also, the kit may further comprise expandable device 60 that may be inserted into a body part. In one embodiment, the expandable device may comprise a first delivery member 68. In an embodiment, the delivery member comprises a distal elongated member 68a and a proximal elongated member 68c. In certain embodiments, the expandable device may comprise a delivery system. In one embodiment, the first and second access members 2, 4 may be used as the delivery system for the expandable device. Also, the second inner member 34 comprising a magnet 36 at the end may also be used to deliver the expandable device where the expandable device includes a ferrule tip 32 (FIG. 20).

In one embodiment, the kit may comprise an inner wrap 97, that may be sealed by heat or vacuum to prevent the components of the kit from being exposed to the outside environment. Also, in one embodiment, the kit may include an outer wrap (not shown) and/or a case 94 to protect the components of the system from physical damage. The inner/outer wraps may be made of wrap materials commonly used in the art such as polyethylene, TYVEK™, or MYLAR™, to allow for visualization of the components in the kit and or sterilization using a sterilizing gas. Sterilization may be by heat, pressure and/or sterilization gas as is known in the art. Also, the kit may include directions for use by a physician or other trained personnel.

Each of the components used in the products, systems, and kits of the present invention may comprise a material that may be sterilized by either chemical treatment, high temperature, and/or high pressure, exposure to sterilizing gas, or a combination of sterilization treatments as is known in the art. Also, the components of the products, systems, and kits of the present invention may be disposable, or may be formulated to allow for cleaning, re-sterilization, and re-use.

It will be understood that each of the elements described above, or two or more together, may also find utility in applications differing from the types described. While the invention has been illustrated and described as devices, systems, kits and methods for multipoint access to an internal body part, it is not intended to be limited to the details shown, since various modifications and substitutions can be made without departing in any way from the spirit of the present invention. Where method and steps describe above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. As such, further modifications and equivalents of the invention herein disclosed may occur to persons skilled in the art using no more than routine experimentation, and all such modifications and equivalents are believed to be within the spirit and scope of the invention as described herein. All patents and published patent applications referred to in this document are incorporated by reference in their entireties as if each individual publication or patent application were specifically and individually put forth herein.

### EXAMPLES

### Example 1

### Vertebral Body Sectioner

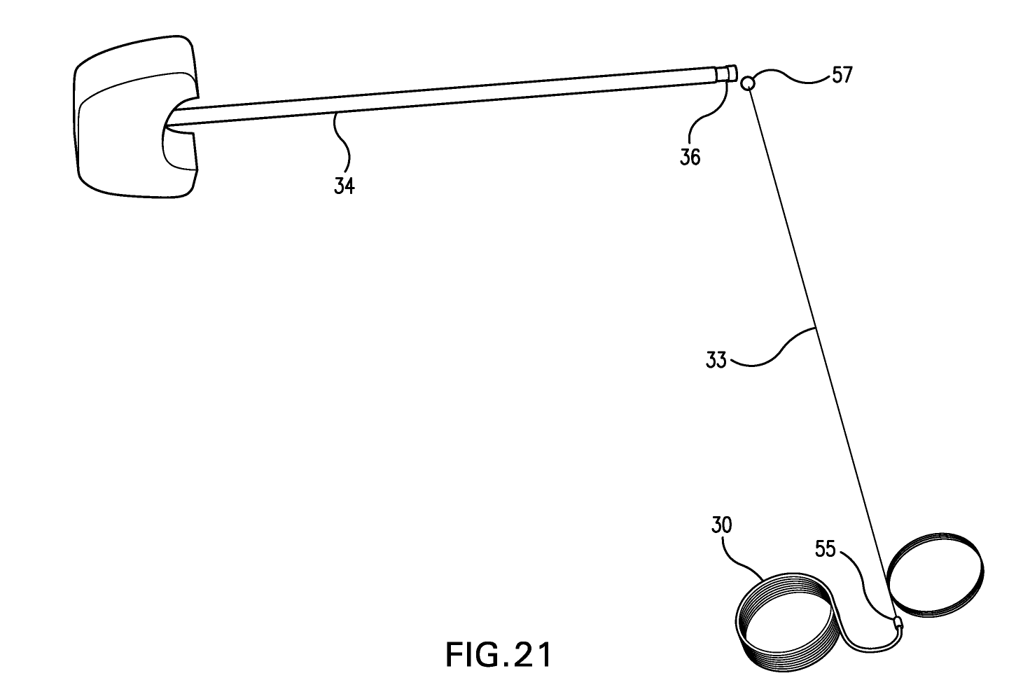

A vertebral body sectioners of the present invention have been made and used on cadavers for testing. Subsequent revisions have embodied design improvements intended to enhance the user's ability to more easily pass the wire saw from the first access member to the second access member. A CAD image of a vertebral body sectioner of the present invention is shown as FIG. 21.

The first inner member was fashioned to include a wire saw assembly. Thus, a lead wire 33 was attached to the end of a giggly saw 30 to facilitate pushing the saw through the cannula. The lead wire consisted of a 0.009″ diameter tungsten leader wire that is attached, in a serial manner, to a 1/32″ diameter stainless steel wire saw. The wires were joined together through the use of a bullet-shaped crimp 55 with a diameter slightly greater than 1/32″. In this assembly, the tungsten leader wire is intended to aid in passing the wire saw assembly between the access members. The leader wire is much thinner and therefore more flexible than the wire saw. This added flexibility allows the leader wire to be manipulated around sharper bends and thus enhances the ease of passing the wire saw assembly between the access members. Also, the wire is less prone to catching on the outer edge of the second access member as it is pulled from one cannula to the next. Once the wire saw has been pulled into the second access member, the wire saw itself can then be easily passed into the second access member by simply pulling on the leader wire and pulling the wire saw along with it through the second cannula.

In addition, at the distal end of the leader wire is a spherical rare earth magnet 57 that had been mechanically bonded onto the leader wire. The use of a rare earth magnet here was intended to strengthen the attractive force between the wire saw assembly (first inner member) and the stylet (second inner member). Since the stylet has a rare earth magnet at its distal end as well, the force of attraction and bonding between the distal ends of bother inner members is strong and ensures that the distal ends will always bond when brought close to one another. The spherical shape of the distal end of the wire saw assembly can enhance the ability of the wire saw assembly to be passed between the access members. The spherical shape is less prone to getting hung up or caught on the outer edge of the second access member as the first inner member is pulled into the second access member by the second inner member.

The second inner member is a stylet 36, similar to the stylet used in the KYPHX® Osteo Introducer™ device that is commercially available from Kyphon, Inc., with a rare earth magnet bonded to its distal end.

### Example 2

### Sectioning A Vertebral Body

The present invention may be used to section and repair a vertebral body or intervertebral disc. Thus, a percutaneous access is used to insert two cannulas into a vertebral body or an intervertebral disc. Next, a first elongated member comprising a wire saw with a ferrule on the distal end is passed through one of the cannulas, and a stylet with a magnet on the distal end is passed through the second cannula. A communication may be established between the magnet and the ferrule allowing the saw to be drawn through the first cannula and into the second cannula. By pulling back and forth on the saw, a portion of the vertebral body may be sectioned.

At this point, the saw may be removed, and two inflatable members (e.g., balloons) are inserted into the portion of the vertebral body that has been cut away. One balloon may be inserted via each cannula. The balloons may be expanded, such that the anterior portion of the vertebral body is lifted up until the endplates of the vertebral body are parallel. At this point, there is a wedge-shaped opening in the vertebral body.

Using the cannulas, an expandable barrier may then be emplaced in the vertebral body. Thus, a first elongated member comprising a ferrule on the distal end and including an expandable barrier is passed through one of the cannulas. A stylet with a magnet on the distal end is passed through the second cannula. A communication may be established between the magnet and the ferrule allowing the barrier to be drawn through the first cannula and positioned in the vertebral body. At this point, the expandable member may be manipulated to expand across the gap previously made in the anterior vertebral wall. A third cannula may then be inserted into the vertebral body (e.g., using a transpedicular access) and a bone repair material emplaced.

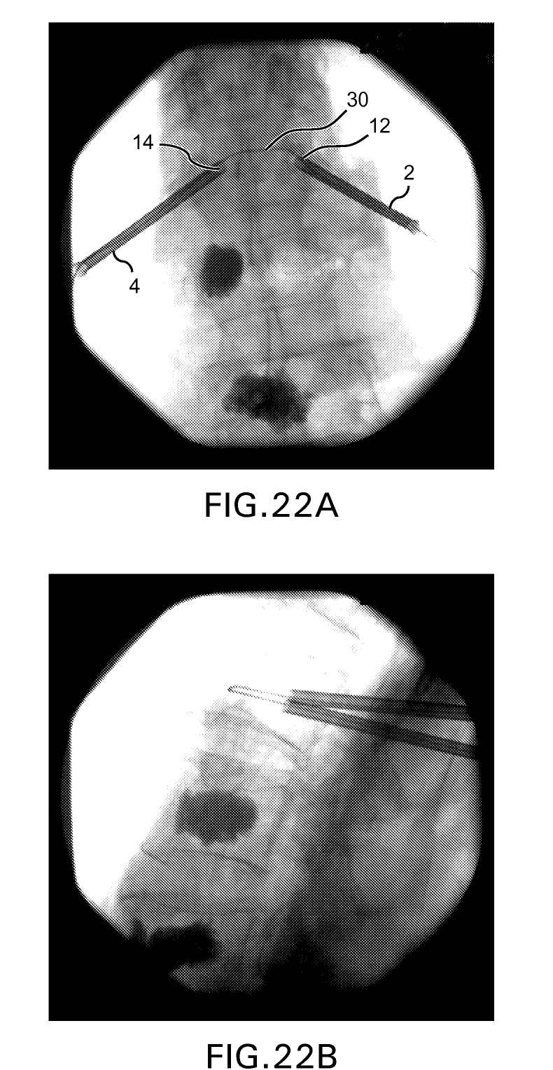

FIG. 22 shows an actual fluoroscopic image of a device for bilateral access of the present invention inserted in a vertebral body. FIG. 22A shows an anterior to posterior (A-P) view, illustrating the two cannulas 2, 4 and the wire inner member 30 positioned within a vertebral body 6. FIG. 22B shows a side view, illustrating the positioning of the cannula tips 12, 14 and the wire saw relative to the anterior wall 5 and posterior wall 7 of the vertebral body 6.

The foregoing is considered as illustrative only of the principal of the invention. Since numerous modifications and changes will readily occur to those skilled in the art, it is not intended to limit the invention to the exact embodiments shown and described, and all suitable modifications and equivalents falling within the scope of the appended claims are deemed within the present inventive concept.

That which is claimed is:

1. A method to access the interior of an internal body part comprising the steps of:
(a) accessing a body part via a first entry point using a first access member;
(b) accessing the body part via a second entry point using a second access member;
(c) positioning the first and second access members such that the distal ends of each access member are in position to communicate with each other; and
(d) establishing a communication between the two access members, such that the two access members function in a coordinated manner.

2. The method of claim 1, wherein the body part is a vertebral body.

3. The method of claim 2, wherein the first access member is inserted through a first pedicle on the vertebral body to access the vertebral body, and the second access member is inserted through a second pedicle on the vertebral body to access the vertebral body.

4. The method of claim 2, wherein the two entry points into the vertebral body are extrapedicular.

5. The method of claim 1, wherein the body part comprises an intervertebral disc.

6. The method of claim 1, wherein the step of establishing a communication comprises:
(i) passing a first inner member through the first access member;
(ii) passing a second inner member through the second access member; and
(iii) engaging the distal end of the first inner member with the distal end of the second inner member.

7. The method of claim 6, wherein the distal end of the first inner member comprises a fixture that is able to bind to a fixture on the distal end of the second inner member.

8. The method of claim 6, wherein the distal end of one of the inner members comprises a magnet, and the distal end of the other inner member comprises a material that binds to the magnet.

9. The method of claim 8, wherein the distal end of the first inner member comprises a ferrous material or a magnet.

10. The method of claim 8, wherein the distal end of the second inner member comprises a magnet.

11. The method of claim 6, wherein the distal end of the first inner member is pulled through the second access member.

12. The method of claim 11, wherein the first inner member is manipulated by an operator to modify the interior of the body part.

13. The method of claim 11, wherein the first inner member is positioned to functionally connect the distal end of the first access member and the distal end of the second access member.

14. The method of claim 11, wherein the first inner member comprises a cutting tool.

15. The method of claim 14, wherein the cutting tool is a wire saw.

16. The method of claim 15, wherein the operator pulls on each end of the wire saw in an alternating motion to cut through a portion of the body part that is positioned between the distal ends of the first and second access members.

17. The method of claim 11, wherein the first inner member comprises an expandable barrier.

18. The method of claim 11, wherein the first inner member comprises an inflatable membrane.

19. A method comprising:
(a) establishing a first percutaneous access to a vertebral body in a subject;
(b) establishing a second percutaneous access to the vertebral body;
(c) passing at least a distal portion of a device into the vertebral body through the first percutaneous path; and
(d) introducing at least a distal end of the device into the distal end of the second percutaneous path.

20. The method of claim 19, wherein the device comprises a cutting tool.

21. The method of claim 20, wherein an operator pulls on each end of the cutting tool in an alternating motion to cut through a portion of the body part that is positioned between the distal ends of the first and second access members.

22. The method of claim 19, wherein the device comprises an expandable member.

23. The method of claim 19, wherein the first and second percutaneous access into the vertebral body is transpedicular.

24. The method of claim 19, wherein the first and second percutaneous access into the vertebral body is extrapedicular.

25. The method of claim 19, wherein the step of introducing at least a distal end of the device into the distal end of the second percutaneous path comprises establishing a connection between the distal end of the device and a member that is inserted into the vertebral body via the second percutaneous path.

26. The method of claim 25, wherein the distal end of the device comprises a portion that binds to the distal end of the member that is inserted into the vertebral body via the second percutaneous path.

27. The method of claim 26, wherein the distal end of the device comprises a material that binds to a magnet, and the distal end of the member that is inserted into the vertebral body via the second percutaneous path comprises a material that binds to a magnet.

28. A method for sectioning at least a portion of a body part comprising:
(a) accessing the body part via a first entry point using a first access member;
(b) accessing the body part via a second entry point using a second access member;
(c) positioning the first and second access members such that the distal ends of each access member are in position to communicate with each other; and
(d) establishing a communication between the two access members, such that the two access members function in a coordinated manner, wherein the step of establishing a communication between the access members comprises: (i) passing a first inner member through the first access member; (ii) passing a second inner member through the second access member; and (iii) engaging the distal end of the first inner member with the distal end of the second inner member.

29. The method of claim 28, wherein the body part is at least one of a vertebral body or an intervertebral disc.

30. The method of claim 29, wherein the first access member is inserted through a first pedicle on the vertebral body to access the vertebral body, and the second access member is inserted through a second pedicle on the vertebral body to access the vertebral body.

31. The method of claim 29, wherein the two entry points into the vertebral body are extrapedicular.

32. The method of claim 28, wherein the first inner member comprises a saw.

33. The method of claim 32, further comprising the steps of:
(e) pulling the distal end of the saw through the second access member; and
(f) pulling on each end of the wire saw in an alternating motion to cut through a portion of the body part positioned between the distal ends of the first and second access members.

34. The method of claim 33, further comprising the steps of: (g) of inserting a balloon into the body part via at least one of the access members; and (h) expanding the balloon to facilitate sectioning of the body part.

35. The method of claim 34, further comprising step (i) of inserting an expandable barrier in the body part via at least one of the access members.

36. The method of claim 20, wherein the step (i) of inserting an expandable barrier in the body part comprises the substeps of: passing a first inner member comprising an expandable barrier through the first access member; passing a second inner member through the second access member; engaging the distal end of the first inner member with the distal end of the second inner member to position the barrier between the distal ends of the first and second access members; and expanding the barrier.

37. The method of claim 21, wherein the expandable barrier comprises an inflatable membrane.

38. A device for multipoint access of a body part comprising:
(a) a first access member;
(b) a second access member;
(c) a first inner member to be inserted into the first access member; and
(d) a second inner member to be inserted into the second access member, wherein the distal end of the first inner member comprises a first binding component that can engage and bind to a second binding component on the distal end of the second inner member.

39. The device of claim 38, wherein the distal end of one of the inner members comprises a magnet, and the distal end of the other inner member comprises a material that binds to the magnet.

40. The device of claim 38, wherein the distal end of at least one inner member comprises a ferrule material.

41. The device of claim 38, wherein the first inner member comprises a tool for repair or modification of the body part.

42. The device of claim 38, wherein the first inner member comprises a cutting tool.

43. The device of claim 38, wherein the first inner member comprises a wire saw.

44. The device of claim 38, wherein the first inner member comprises an expandable member.

45. The device of claim 38, wherein the second inner member comprises a stylet.

46. The device of claim 38, wherein the first and second access members are configured for accessing a vertebral body.

47. The device of claim 46, wherein the first and second access members are configured for accessing the vertebral body by percutaneous access.

48. The device of claim 38, wherein the first access member is configured for accessing interior of a vertebral body through a first pedicle of the vertebral body, and the second access member is configured for accessing interior of the vertebral body through a second pedicle of the vertebral body.

49. The device of claim 38, wherein the first and second access members are configured for accessing the interior of a vertebral body by an extrapedicular access.

50. The device of claim 38, wherein the first and second access members are configured for accessing an intervertebral disc.

51. The device of claim 50, wherein the first and second access members are configured for percutaneous access.
